SEQUENCE LISTING (1 sequence)

SEQ ID NO: 1 - protein, 8 aa, Homo sapiens
GKVQIVYK

(19) United States
(12) Patent Application Publication
(10) Pub. No.: US 2005/0048539 A1
(43) Pub. Date: Mar. 3, 2005

(54) Methods to monitor molecule conformation and molecule/molecule proximity

(75) Inventors: Bradley T. Hyman, Charlestown, MA (US); Oksana Berezovska, Charlestown, MA (US); Brian Bacskai, Charlestown, MA (US); Alberto Lleo, Charlestown, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: The General Hospital Corporation, Boston, MA (US)

(21) Appl. No.: 10/868,756

(22) Filed: Jun. 14, 2004

Related U.S. Application Data
(60) Provisional application No. 60/478,642, filed on Jun. 13, 2003.

(51) Int. Cl. C12Q 1/68; G01N 33/53; G06K 9/00

(52) U.S. Cl. 435/6; 435/7.1; 382/128

(57) ABSTRACT

The invention relates in part to methods for monitoring the conformation of molecules, include proteins. The methods of the invention are also useful to monitor the distance between two or more molecules, such as the distance between two proteins in a cell. Additionally, the methods of the invention are useful for determining the location of a molecule, e.g. a protein, within a cell or other environment. The invention also relates in part to assays for identifying and testing candidate compounds for modulating molecule conformation and/or molecule interactions.

### RELATED APPLICATION

This application claims priority under 35 U.S.C. §119 from U.S. provisional application Ser. No. 60/478,642, filed: Jun. 13, 2003, the content of which is hereby incorporated herein in its entirety.

### GOVERNMENT SUPPORT

This invention was made with government support under NIH P01 grant number AG15379 and grant number 5R01EB000768. The government has certain rights in this invention.

### FIELD OF THE INVENTION

The invention relates in part to methods for monitoring the conformation of molecules, include proteins. The invention also relates in part to methods to monitor the distance between two or more molecules, such as the distance between two proteins. Methods of the invention are also useful for determining the location of a molecule, e.g. a protein, within a cell or other environment. The invention also relates in part to assays that are useful for identifying and testing candidate compounds for modulating molecule conformations and/or modulating the distance between of two or more molecules.

### BACKGROUND OF THE INVENTION

Immunofluorescence is a well-established and powerful technique to demonstrate the spatial distribution of a protein of interest within single cells or complex biological tissue. Double immunofluorescence with spectrally distinct fluorophores can also determine whether a pair of proteins co-localize, with a resolution limited by the wavelengths of light used for the measurements, or approximately 0.5 μm. Antibodies provide very high specificity, and fluorescence measurements exhibit high sensitivity, with low background. Therefore, measurements of fluorescence intensities have proven highly useful for characterization of the spatial localization of specific proteins within biological cells or tissues. By exploiting fluorescence resonance energy transfer (FRET), the spatial resolution of co-localization of two fluorophores is increased to the scale of 5 nanometers (Lakowicz, J. R., Principles of Fluorescence Spectroscopy. 2nd ed. 1999). FRET depends on the close physical interaction of two fluorophores, a donor and acceptor, dependent on the distance between them to the sixth power, and does not occur if this distance exceeds ˜10 nm (Lakowicz, J. R., Principles of Fluorescence Spectroscopy. 2nd ed. 1999). The fluorophores also need to fulfill two other requirements; the emission spectrum of the donor must overlap with the excitation spectrum of the acceptor, and the dipoles of the molecules must be oriented appropriately to allow energy transfer (Lakowicz, J. R., Principles of Fluorescence Spectroscopy. 2nd ed. 1999). These requirements are relatively easy to achieve, and measurements of FRET can be performed using fluorescein as the donor and rhodamine as the acceptor.

FRET can be detected in several ways. Using standard fluorescence intensity measures, FRET is characterized by a quenching of the donor fluorescence with a corresponding emission of acceptor fluorescence, all while exciting at the donor absorption peak. In ideal cases, these measures would be large and interpretable, but in reality the signals are small and plagued by interference from crosstalk between direct excitation of the acceptor at the donor excitation wavelengths and emission of the donor extending to the acceptor emission wavelengths (Gordon, G. W. et al., Biophys J 74: 2702-2713, 1998). The most convincing measure of FRET in spatially interesting biological tissues involves photobleaching the acceptor fluorophore independently of the donor, and measuring the resultant de-quenching of the donor fluorescence that resulted from FRET (Lakowicz, J. R., Principles of Fluorescence Spectroscopy. 2nd ed. 1999; Wouters, F. S. et al., Curr Biol. 9(19): 1127-1130, 1999; McLean, P. J. et al., J Biol Chem. 275(12): 8812-8816, 2000). This technique is reliable, but makes spatially distinct measurements difficult and tedious, and is destructive. This technique cannot be performed on dynamic processes occurring in living cells or tissue.

An alternative to measurements of FRET based on fluorescence intensity depends on fluorescence lifetime determinations. Lifetimes are the average time a fluorophore spends in the excited state before emitting a photon and returning to the ground state, and are measured in nanoseconds (Lakowicz, J. R., Principles of Fluorescence Spectroscopy. 2nd ed. 1999; Emptage, N. J., Curr Opin Pharmacol. 1(5):521-525, 2001; Hanley, Q. S. et al., Cytometry 43(4): 248-260, 2001). The fluorescence lifetime of a fluorophore is affected by its local microenviromnent, and by FRET. In the presence of a suitable acceptor within a distance for FRET, the lifetime of a donor's fluorescence will decrease, and can be measured quantitatively (Bastiaens, P. I. et al., Trends Cell Biol. 9(2): 48-52, 1999; Verveer, P. J. et al., J Microsc. 202(Pt 3): 451-456, 2001). The decrease in lifetime is proportional to the distance between donor and acceptor. Fluorescence lifetime imaging microscopy (FLIM) allows quantitative determinations of the distance between a donor and acceptor fluorophore on the scale of nanometers, but with the added benefit of microscopic imaging within cells or tissue, to pinpoint where in a biological tissue, with the resolution of the light microscope, that FRET is occurring.

The previously known methods of FRET and FLIM imaging are useful for analysis of molecule interactions but lack sensitivity in that they lack an ability to spatially quantify the fluorescence donor-labeled molecules undergoing the transfer of fluorescence energy. This drawback limits the resolution and sensitivity of current FRET and FLIM methods. To accurately determine intra- and inter-molecule characteristics, conformations, and distances between two or more molecules of interest, more sensitive molecule analysis and monitoring methods are desirable.

### SUMMARY OF THE INVENTION

We have developed a method of monitoring the distance between two or more molecules and have also devised a method of monitoring the conformation of molecules. Both methods involve the use of techniques utilized in fluorescence resonance energy transfer (FRET) and in fluorescence lifetime imaging microscopy (FLIM) in combination with a novel method of resolving both the degree of FRET and the proportion of FRET in a spatially resolved way.

We have developed methods that utilize FLIM measurements. The methods include, in part, labeling molecules of interest with either a donor or acceptor fluorophore or a combination of a donor and acceptor fluorophore, exposing the molecules to an excitation source, and determining the lifetime of the donor fluorophore fluorescence measured. A decrease in the fluorescence lifetime of the donor fluorophore is proportional to the distance between the donor and acceptor fluorophores. We have utilized methods of the invention to examine the distance between molecules. For example, the methods of the invention have been used to examine the distance between PS1 and amyloid precursor protein in neuronal cells and to investigate the effects of various agents, e.g. agents that disrupt nicastrin, on the distance between PS1 and APP (see Example 2).

The methods of the invention are also useful to assess the conformation of a individual molecule. Aspects of this method of the invention involve labeling different regions of a single molecule of interest with a donor and an acceptor fluorophore, exposing the labeled molecule to an excitation source, and determining the lifetime of the donor fluorophore fluorescence. The fluorescence lifetime of the fluorophore donor is proportional to the distance between the donor and acceptor fluorophores, and the lifetime value can be used to evaluate conformation of a molecule. The methods of the invention are also useful to assess changes in the conformation of a molecule of interest and can be used to assess the cause of conformational changes as well as the effect of conformational changes on function of a molecule of interest. We have utilized methods of the invention to examine the conformation of proteins. For example, using the methods of the invention, aspects of the conformation of β amyloid (Aβ) have been examined (see Example 1).

The methods of the invention also include, in part, measurements of donor fluorescence in the time domain. Time-domain measurements permit the proportion of molecules displaying FRET to be quantified within a microscopic region. The claimed methods allow a reliable determination of the fraction of FRETing to non-FRETing molecules within a sample on a pixel-by-pixel basis. This is a significant advantage in using our novel time-domain FLIM for FRET measurements as compared with other FRET determinations, which cannot discriminate between weakly associating fluorophore pairs, or a mixed population of strongly associating and non-associating pairs. The methods of the invention allow assessment and monitoring of molecule conformations and proximity and/or associations in living cells and in non-living (e.g. fixed) cells and tissues.

Additionally, the methods of the invention can also be used to test candidate agents to determine whether such agents effect the conformation of a molecule, (e.g. a protein) and/or whether the agent effects the distance between two or more molecules (e.g. proteins).

According to one aspect of the invention, methods of assessing the distance between two molecules are provided. The methods include: obtaining a biological sample comprising a first molecule labeled with a donor fluorophore and a second molecule labeled with a acceptor fluorophore, exposing the biological sample to a pulsed excitation source with a predetermined pulse rate and pulse frequency, measuring the fluorescence lifetime of the donor fluorophore, calculating the distance between the donor and acceptor fluorophores as a measure of the distance between the first and second molecules, wherein the length of the fluorescence lifetime correlates with the distance between the first and second molecules, spatially resolving the proportion of donor fluorophore that transfers fluorescence energy, and optionally imaging the biological sample. In some embodiments, the imaging is light microscopy imaging. In some embodiments, the molecules are proteins. In some embodiments, the biological sample comprises cells. In some embodiments, the cells are transformed cells. In some embodiments, the cells are live cells. In some embodiments, the cells are fixed cells. In some embodiments, the cells are neuronal cells. In some embodiments, first and second proteins are selected from the group consisting of: presenilin 1, presenilin 2, nicastrin, Aph-1, Pen-2, secretase complex proteins, APP and β-amyloid (Aβ). In some embodiments, the first and second proteins are presenilin 1 and APP. In some embodiments, the donor fluorophore has an emission spectrum that overlaps with the excitation spectrum of the acceptor fluorophore. In some embodiments, the donor fluorophore is selected from the group that includes: fluorescein isothiocyanate (FITC), Alexa 488, Oregon Green 514, and Oregon Green 488. In some embodiments, the donor fluorophore is fluorescein isothiocyanate (FITC). In some embodiments, the acceptor fluorophore is selected from the group consisting of: Cy3, Rhodamine, Texas Red, and Alexa 568. In certain embodiments, the acceptor fluorophore is Cy3. In some embodiments, the molecules are labeled using a means selected from the group consisting of: antibody labeling, derivatization, chemical modification, and genetic engineering. In some embodiments, the pulsed excitation source is a laser. In some embodiments, the laser is a Ti-sapphire laser. In some embodiments, the predetermined pulse length is from about 1 femtosecond to about 1 picosecond. In some embodiments, the predetermined pulse length is about 1 femtosecond. In some embodiments, the predetermined pulse length is about 10 femtoseconds. In some embodiments, the predetermined pulse length is about 100 femtoseconds. In some embodiments, the predetermined pulse frequency is from about one pulse per 1 nanosecond to about 1 pulse per 100 nanoseconds. In some embodiments, the predetermined pulse frequency is about one pulse per 12 nanoseconds.

According to yet another aspect of the invention, methods of screening a candidate pharmaceutical agent for an effect on the distance between two molecules are provided. The methods include contacting a first biological sample with a candidate pharmaceutical agent and assessing the distance between a first and second molecule in the sample as in any of the methods of the foregoing aspect of the invention, assessing the distance between a first and second molecule in a second biological sample as in any of the methods of the foregoing aspect of the invention, wherein the second biological sample is a sample not contacted with the candidate pharmaceutical agent and the first and second molecules are of the same type as the first and second molecules in the first biological sample, comparing the distance between the first and second molecules in the first biological sample and between the first and second molecules in the second biological sample as an indication of the effect of the candidate pharmaceutical agent on the distance between the first and second molecules.

According to yet another aspect of the invention, methods of determining a molecule's conformation are provided. The methods include obtaining a biological sample comprising a molecule labeled in a first region with a donor fluorophore and labeled in second region with an acceptor fluorophore, exposing the biological sample to a pulsed excitation source with a predetermined pulse rate and pulse frequency, measuring the fluorescence lifetime of the donor fluorophore, and calculating the distance between the donor and acceptor fluorophores as a determination of the conformation of the molecule, wherein the length of the fluorescence lifetime correlates with the distance between the first and second molecule regions, spatially resolving the proportion of donor fluorophore that transfers fluorescence energy, and optionally imaging the biological sample. In some embodiments, the imaging is light microscopy imaging. In some embodiments, the molecule is a protein. In some embodiments, the biological sample comprises cells. In some embodiments, the cells are transformed cells. In some embodiments, the cells are live cells. In some embodiments, the cells are fixed cells. In some embodiments, the cells are neuronal cells. In some embodiments, the protein is selected from the group consisting of: presenilin 1, presenilin 2, nicastrin, Aph-1, Pen-2, secretase complex proteins, APP and β-amyloid (Aβ). In some embodiments, the protein is presenilin 1. In some embodiments, the first and second regions are selected from N-terminal region, mid-region or C-terminal region of the protein. In some embodiments, the donor fluorophore has an emission spectrum that overlaps with the excitation spectrum of the acceptor fluorophore. In some embodiments, the donor fluorophore is selected from the group that includes: fluorescein isothiocyanate (FITC), Alexa 488, Oregon Green 514, and Oregon Green 488. In some embodiments, the donor fluorophore is fluorescein isothiocyanate (FITC). In some embodiments, the acceptor fluorophore is selected from the group consisting of: Cy3, Rhodamine, Texas Red, and Alexa 568. In some embodiments, the acceptor fluorophore is Cy3. In some embodiments, the molecule regions are labeled using a means selected from the group consisting of: antibody labeling, derivatization, chemical modification, and genetic engineering. In some embodiments, the pulsed excitation source is a laser. In some embodiments, the laser is a Ti-sapphire laser. In some embodiments, the predetermined pulse length is from about 1 femtosecond to about 1 picosecond. In some embodiments, the predetermined pulse length is about 1 femtosecond. In some embodiments, the predetermined pulse length is about 10 femtoseconds. In some embodiments, the predetermined pulse length is about 100 femtoseconds. In some embodiments, the predetermined pulse frequency is from about one pulse per 1 nanosecond to about 1 pulse per 100 nanoseconds. In some embodiments, the predetermined pulse frequency is about one pulse per 12 nanoseconds.

According to another aspect of the invention, a method of screening a candidate pharmaceutical agent for an effect on the conformation of a molecule is provided. The method includes contacting a first biological sample with a candidate pharmaceutical agent and assessing the conformation of a molecule using any of methods of the foregoing aspect of the invention, assessing the conformation of the molecule in a second biological sample using the any of the methods of the foregoing aspect of the invention, wherein the second biological sample is a sample not contacted with the candidate pharmaceutical agent and the molecule is the same type of molecule as the molecule in the first biological sample, comparing the conformation of the molecule in the first biological sample and conformation of the molecule in the second biological sample as an indication of the effect of the candidate pharmaceutical agent on the conformation of the molecule.

### BRIEF DESCRIPTION OF THE DRAWINGS

FIG. 1 is a Schematic diagram of the FLIM acquisition system.

FIG. 2 shows digitized photomicrographic images of results of FRET with CFP and YFP that is detectable with FLIM. FIG. 2A shows cells expressing CFP alone, which had a single lifetime component of 2.9±0.1 ns. FIG. 2B shows results with YFP, which is not excited with multiphoton excitation at 740 nm. FIG. 2C shows results from CFP contransfected with YFP, which yields a fluorescence lifetime indistinguishable from CFP alone-transfected cells, 2.9±0.1 ns, indicating that there is no FRET between these individual proteins. However, FIG. 2D shows that when CFP is fused to YFP in the same construct, the fluorophores are forced into close proximity, and the lifetime of CFP is reduced in these cells to 1.9±0.1 ns, indicative of FRET. Scale bar=20 mm.

FIG. 3 is a graph depicting the pH dependence of fluorescein and fluorescein-labeled IgG antibody. The free fluorescein solutions (circles) have longer lifetimes than the fluorescein-labeled antibody (squares), and each show a weak dependence on pH, with the lifetimes higher at elevated pH.

FIG. 4 is a graph depicting fluorescence lifetime dependence of labeled streptavidin complex. Solutions of DTAF-streptavidin (Jackson Immunologicals) DTAF-streptavidin with free biotin, and DTAF-streptavidin in the presence of biotinylated antibody were placed in glass capillary tubes at 0.1 mg/ml in PBS. Fluorescence lifetime images were acquired for 3 min, using 800 nm excitation, and a green emission filter (515df30). Average values were determined from within each sample. (p<0.05, Student's t-test).

FIG. 5 is a graph showing a calibration curve for mixed populations of FRET and non-FRET pairs. The calibration curve plots the calculated amplitude of the fast lifetime component as a function of the fraction of doubly labeled peptide in the sample mixtures. The results demonstrate that the curve-fitting routine reliably estimates the fraction of FRETing to non-FRETing molecules within a sample, on a pixel-by-pixel basis.

FIG. 6 shows digitized photomicrographic images demonstrating that senile plaques like those found in Alzheimer's disease are morphologically nonuniform. FIG. 6A shows that the fluorescence lifetime of FITC-10d5 immunolabeling is uniform across the plaques, with a mean lifetime of 2.6±0.1 ns. FIG. 6B illustrates a representative example of a plaque that is doubly labeled with FITC-10d5 and rhodamine-3d6. In this example, FRET is observed throughout the plaque, as evidenced by the reduction in the fluorescence lifetime of fluorescein to 0.9±0.1 ns. The less intense brightness in the core represents longer lifetimes, and less FRET than the fringes. Scale bar=20 μm.

FIG. 7 shows diagrams, graphs, and digitized photomicrographic images of results of control experiments. FIG. 7A shows images of the negative (FITC only) and positive (CY3-anti-FITC) controls. If molecules are closer to each other, donor fluorescence (FITC) lifetime is shorter, and the color will be closer to red. The graphs show lifetime distribution collected for every pixel of the images: positive control shows shift to the left. FIG. 7B shows the Cy-3 acceptor in one-half of the cell (positive control) was destroyed by photobleaching (outlined area), leading to dequenching of the FITC fluorescence intensity and a shift to a longer lifetime.

FIG. 8 shows digitized photomicrographic confocal microscope images of cells double immunostained with FITC-labeled PS1 (FIG. 8A) and CY3-labeled APP (FIG. 8B) antibodies. The results demonstrate predominantly perinuclear localization of the proteins. Bar=10 mm.

FIG. 9 provides digitized photomicrographic images and results of FLIM analysis of the proximity between APP and PS1 molecules within the cell. FIG. 9A shows intensity image showing a standard immunostaining pattern for PS1, similar to that shown in FIG. 8A. FIG. 9B shows a color-coded FLIM image showing lifetimes, reflecting proximity between PS1 and APP. The cell regions showing closest proximity between PS1 and APP are in the distal compartments, near the cell surface. Colorimetric scale shows fluorescence lifetime in picoseconds. FIG. 9C and FIG. 9D show enlarged boxed areas from FIG. 9B. FLIM image is superimposed onto a table with calculated average lifetimes for each pixel of the image. Magnification bar=0.2 mm.

FIG. 10 shows digitized photomicrographs indicating results of photobleach dequenching FRET between APP and PS1, which demonstrates close proximity between C-terminus of APP and loop region of PS1. FIG. 10 shows CY3-labeled APP (568 nm emission) before and after photobleaching the acceptor (CY3) (FIGS. 10A and B, respectively) and FITC labeled PS1 (488 nm emission) before and after photobleaching the acceptor (CY3) (FIGS. 10C and D, respectively) in a selected area within the cell. FIG. 10 also demonstrates that DAPT does not prevent close association of APP with PS1. FIGS. 10E-H show DAPT with CY3-labeled APP (568 nm emission) before and after photobleaching the acceptor (CY3) (FIGS. 10E and F, respectively) and FITC labeled PS1 (488 nm emission) before and after photobleaching the acceptor (CY3) (FIGS. 10G and H, respectively) in a selected area within the cell. Bar=10 mm.

FIG. 11 shows digitized photomicrographic confocal microscope images of wt PS1 (FIG. 11A and FIG. 11B) and aspartate mutant (D257A) PS1 (FIG. 11C and FIG. 11D) double-stained with biotinylated γ-secretase inhibitor WPE-31C-bi (FIG. 11A and FIG. 11C), and PS1 antibody (FIG. 11B and FIG. 11D). PS1 immunostaining co-localizes with CY3-streptavidin labeled WPE-31C-bi in the cells expressing wt PS1. WPE-31C-bi does not bind to aspartate mutant PS1 holoprotein. Bar=20 mm.

FIG. 12 shows a digitized image of a Western blot (FIG. 12A) demonstrating that Nct RNAi leads to a significant inhibition of Nicastrin expression in the cells. The table in FIG. 12B illustrates that a decrease in the FITC-(PS1) lifetime in APP-PS1 double immunostained cells is observed in mock treated cells, indicating a close proximity between the two proteins. This association is eliminated by Nct RNAi treatment: because the FITC lifetime becomes the same as in the PS1-FITC alone control.

FIG. 13 shows a schematic representation of the predicted PS1 conformation for immature PS1 holoprotein (FIG. 13A), and functionally active PS1 heterodimer (FIG. 13B and C), which exists in a dynamic state. For simplicity, only one component of the multimeric α-secretase complex is shown.

FIG. 14 is a bar graph indicating the effects of NSAIDs on Aβ secretion. PS70 cells were treated 24 hours with different NSAIDs and Aβ₄₀, Aβ₄₂ and Aβ₁₋ₓ levels were determined using ELISA. We observed a marked reduction predominantly in Aβ₄₂ after treatment with S-ibuprofen, flurbiprofen, R-ibuprofen and indomethacin but not with aspirin, meloxicam or naproxen. No change in the levels of Aβ₁₋ₓ was observed compared to a vehicle treatment. No cytotoxicity was observed at the doses used assessed by measurement of adenylate kinase levels in the conditioned media. * One-way ANOVA, P<0.05. Black bars=Aβ40, Grey bars=Aβ42, and White bars=Aβ1-x.

FIG. 15 is a bar graph that indicates the effects of NSAIDs on Aβ secretion. PS70 cells were treated 24 hours with different NSAIDs and Aβ₄₀ and Aβ₄₂ levels were determined using ELISA. We observed a dose-dependent reduction, predominantly in Aβ42, after treatment with S-ibuprofen, flurbiprofen, R-ibuprofen and indomethacin but not with meloxicam or naproxen. No cytotoxicity was observed at the doses used assessed by measurement of adenylate kinase levels in the conditioned media. *One-way ANOVA, P<0.05.

FIG. 16 depicts a digitized image of a western blot and a bar graph that indicate NSAIDs do not inhibit BACE or γ-secretase. FIG. 16A shows results of Western blot analysis of the expression of full-length APP, BACE1 and APP CTFs in mouse neuroblastoma cells transfected with human APP751 and treated with different NSAIDs. No change in the levels of expression of full length APP, BACE1 or APP CTFs was detected in cells treated with NSAIDs. As expected, treatment with 1 μM DAPT led to an accumulation of APP CTFs. FIG. 16B shows results of HEK cells treated 24 h with different NSAIDs. Cells were transfected with the plasmids APP-Gal4, pG5E1B-luc, μ-galactosidase, and Fe65, and treated 24 hours with different NSAIDs. After normalization to β-galactosidase activity, no difference in luciferase activity was observed between cells treated with DMSO or NSAIDs.

FIG. 17 is a bar graph that indicates that BACE activity was not reduced in mice brains treated with S-ibuprofen. BACE activity was measured in the brain of mice treated with S-ibuprofen or a control diet using a BACE-specific enzymatic activity assay. No differences were observed in BACE activity in mice treated with S-ibuprofen compared to that of controls.

FIG. 18 is a schematic diagram of a model for the effect of NSAIDs on presenilin conformation. In FIG. 18A PS1 is shown as a ring-like structure that cleaves APP/C99 through the two conserved aspartates (black dots) located in TM6 and 7, liberating Aβ₄₂. FIG. 18B shows that in the presence of NSAIDs, the distance between the N- and C-termini of PS1 increases. This change in the conformation of PS1 shifts the cleavage of APP/C99 towards shorter Aβ species like Aβ_{38.}

### DESCRIPTION OF THE SEQUENCE

SEQ ID NO:1 GKVQIVYK is a sequence homologous to a region of the human tau protein.

### DETAILED DESCRIPTION OF THE INVENTION

The methods of the invention relate, in part, to monitoring the distance between two or more molecules. Additionally, some aspects of the invention relate to methods of monitoring the conformation of a molecule. Both methods involve the use of techniques utilized in fluorescence resonance energy transfer (FRET) and in fluorescence lifetime imaging microscopy (FLIM) in combination with a novel method of resolving both the degree of FRET and the proportion of FRET in a spatially resolved way.

As used herein, the term “molecules” means any type of molecule including, but not limited to: proteins, polypeptides, amino acids, polynucleotides, carbohydrates, lipids, small molecules, or pharmaceutical agents, chemicals, etc. In some embodiments, the two or more molecules monitored using the methods of the invention are both the same class of molecules, e.g. two or more different proteins, or two or more different carbohydrates. In some embodiments, the two or more molecules are in different classes of molecules, e.g. one may be a protein, and the other may be a polynucleotide, or one may be a protein and the other may be a carbohydrate etc. In preferred embodiments, the molecules of the invention are proteins. As used herein the term “molecule of interest” means the molecule to be monitored using the methods of the invention.

As described above, the methods of the invention include the use of donor and acceptor fluorophores. The donor and acceptor fluorophores may be attached to the molecule of interest using a variety of methods including the attachment of the fluorophores to antibodies that specifically bind to the molecule, or molecules, of interest. Thus, in some embodiments, antibody labeled donor and antibody labeled acceptor fluorophores are utilized. In some embodiments, the antibodies may specifically bind to different molecule of interests, e.g. presenilin1 (PS1) and amyloid precursor protein (APP). For example, an antibody-labeled donor may be an antibody that selectively binds to PS1 and an antibody-labeled acceptor may be an antibody that selectively binds to APP.

In other embodiments, two antibodies that bind to different regions of a single protein are labeled, one with a fluorescence donor and the other with a fluorescence acceptor. The antibodies selectively bind to different regions of a single protein molecule and the methods of the invention can be used to assess the conformation of the molecule based on the FRET activity resulting from the proximity of the donor and acceptor fluorophores as determined with the methods of the invention. It will be understood that one region of a protein is different and distinct from another region of the protein if the two regions can be distinguished from each other using the methods of the invention.

In some embodiments of the invention, the region of a protein may an N-terminal region, mid-region, or C-terminal region of the protein. As used herein, the term “region” is used to describe a location in the amino acid sequence of the protein. For example, the N-terminal region of a protein begins at the N-terminus and can include up to about a third of the length of the protein's amino acid sequence. Similarly, the “mid-region” of a protein begins approximately at the center of the protein's amino acid sequence and can include up to about one third of length of the protein's amino acid sequence. The C-terminal region of a protein may end at the C-terminus of the protein and can include up to about a third of the length of the protein's amino acid sequence of the protein.

In some embodiments of the invention, determining the degree of FRET in the sample includes measuring the lifetime fluorescence of the donor fluorophore. The fluorescence lifetime of a fluorophore is affected by its local microenvironment, and by FRET. In the presence of a suitable acceptor within a distance for FRET, the lifetime of a donor's fluorescence will decrease, and can be measured quantitatively. The decrease in lifetime is proportional to the distance between donor and acceptor. The fluorescence lifetime imaging microscopy (FLIM) methods of the invention allow quantitative determination of the distance between a donor and acceptor fluorophore on the scale of nanometers, and with the ability to microscopically image within cells or tissues to identify the location where the FRET is occurring within the biological sample with the resolution of a light microscope. In addition, in some embodiments, the invention also includes the use of FLIM measurements in the time domain, which permits quantifying the proportion of molecules displaying FRET within a microscopic region. The time-domain measurement methods of the invention allow high spatial resolution of the samples. Thus, the methods of the invention permit resolution of both the degree of FRET and the proportion of FRET in a spatially resolved way.

The terms “donor” and “acceptor” are used broadly to encompass traditional donors and acceptors. In FRET, the “donor fluorophore” and the “acceptor fluorophore” are selected so that the donor and acceptor exhibit FRET when the donor moiety is excited. One factor to be considered in choosing the donor/acceptor fluorophore pair is the efficiency of FRET between the two fluorophores. In some embodiments, the efficiency of FRET between the donor and acceptor moieties is at least 10%, in some embodiments, at least 50%, and in other embodiments, at least 80%. The efficiency of FRET can be tested empirically using the methods described herein and known in the art. In the methods of the invention, the donor fluorophore has an emission spectrum that overlaps with an excitation spectrum of the acceptor fluorophore.

FRET is the transfer of photonic energy between fluorophores and is a tool for characterizing molecular detail because it allows determination of changes in distance between the donor and acceptor fluorophores. The methods of the invention include the incorporation of the donor and acceptor fluorophores onto the molecule(s) of interest. The incorporation is the manner in which the fluorophore is attached to a molecule of interest. Methods to incorporate a donor fluorophore and/or an acceptor fluorophore may include: antibody labeling (as noted above), derivatization, chemical modification (including but not limited to covalent attachment), genetic engineering of, or other attachment method known to those of skill in the art that can be used to attach a fluorophore to a molecule.

The high resolution of FRET has been used in many studies of molecular dynamics and biophysical phenomena. Additional information relating to FRET methods can be found in Forster, T. Ann. Physik 2:55-75 (1948). Tables of spectral overlap integrals are also available (for example, Berlman, I. B. Energy transfer parameters of aromatic compounds, Academic Press, New York and London (1973)). FRET is a nondestructive spectroscopic method that can monitor proximity and relative angular orientation of fluorophores in living cells. See, for example, Adams, S. R., et al., Nature 349:694-697 (1991), and Gonzalez, J. & Tsien, R. Y. Biophy. J. 69:1272-1280 (1995).

To undergo FRET, the emission spectrum of the donor overlaps with the excitation spectrum of the acceptor. In some embodiments, a laser is tuned to the excitation wavelength of the donor fluorophore. The donor fluorophore emits its characteristic wavelength and the fluorescence lifetime of the donor correlates with the distance between the donor and acceptor fluorophores. As the acceptor fluorophore moves into interactive proximity with the donor fluorophore, the acceptor fluorophore is excited by the energy from the donor fluorophore. The consequence of this interaction is that the emission of the donor fluorophore may be shortened.

Once a FRET signal is generated it can then be detected and the detected signals from FRET may be analyzed in real time and/or stored in a database for analysis. Embodiments and examples of detection and analysis of the FRET signals that are useful in the methods of the invention are described in the Examples section. Alternative detection and imaging devices known to those of skill in the art may also be used in the methods of the invention. After the detectable signals are generated and detected the signals can be analyzed to determine molecule conformation and molecule-molecule distance information from the cell or sample.

Various factors may be balanced to optimize the efficiency and detectability of FRET methods of the invention. The emission spectrum of the donor fluorescent moiety should overlap as much as possible with the excitation spectrum of the acceptor fluorescent moiety to maximize the signal.

Changes in the distance between two molecules that results from a candidate pharmacological agent can be determined by monitoring FRET at a first and second time, e.g., monitoring before and after contact between the sample containing the molecules of interest that are labeled with donor and acceptor fluorophores of the invention with a candidate pharmacological agent. Using the methods described herein, one can determine the difference in the degree of FRET between the contacted and a non-contacted sample and calculate the effect of the distance between the proteins that results from the candidate compound.

Numerous fluorescent donor and acceptor may be used in methods of the invention, including proteins that fluoresce due to intramolecular rearrangements or the addition of cofactors that promote fluorescence. In the claimed invention examples of fluorescent moieties are fluorescein isothiocyanate (FITC), Alexa 488, Oregon Green 514, Oregon Green 488, Cy3, Rhodamine, Texas Red, Alexa 568. It will be understood by one of ordinary skill in the art that numerous other fluorophores are suitable for use in the methods of the invention.

In the methods of the invention, the donor fluorophore is excited by the appropriate excitation wavelength for that fluorophore. Suitable values for the length, duration, and frequency for excitation source used to excite a donor fluorophore of the invention are provided herein and additional values are known by those of ordinary skill in the art, or can be determined without undue experimentation. In some embodiments, a Ti:sapphire laser with 100 femtosecond pulses at 82 MHz can be used as the excitation source. In certain embodiments, picosecond pulses are used, and in some embodiments, pulse frequencies down to kilohertz are used. In general, it is preferable for the pulse widths to be shorter than 500, 400, 300, 200, or 100 picoseconds. Additionally, it is preferable for the pulse frequency to be less than 82 MHz. In some embodiments, pulsed diode lasers that run at 50-80 MHz, with pulse widths of about 100-200 ps, are used in the methods of the invention. The Ti:sapphire laser may be used for 2-photon excitation at near infrared wavelengths. The diode lasers at wavelengths of about 750-800 nm may be useful for 2-photon excitation, and in some embodiments, one-photon excitation with a diode laser at 450 nm is used. One of ordinary skill in the art will recognize that numerous donor and acceptor fluorophores and excitation sources and parameters can be used in the claimed invention and will be able to determine combinations of such fluorophores and the appropriate excitation wavelengths without undue experimentation.

In some embodiments, the methods of the invention are useful to localize a molecule to a cellular region. For example, a donor fluorophore may be attached to a cell component and the acceptor fluorophore attached to a molecule of interest. Examples of cell structure molecules (proteins) that can be utilized in methods of the invention include, but are not limited to, a receptor ligand, a nuclear localization sequence (NLS), a plasma membrane targeting signal, or any other region-specific protein. Other cell or tissue region-specific proteins are known to those skilled in the art, or can be readily ascertained without undue experimentation.

The introduction of donor and acceptor fluorophore labels onto molecules of interest in cells allows those of skill in the art to monitor the spatial location of the molecules, the conformation of the molecules, and the distance between molecules of interest in those cells. Additionally, use of the monitoring methods of the invention allows diagnosis of disorders, such as Alzheimer's or other disorders. For example, such disorders can be identified by abnormal protein conformation, protein localization and/or distance between two or more proteins in cell samples. The term “abnormal” refers to a conformation that differs from that found in a disease-free cell or sample. Additionally, an “abnormal” distance between two or more proteins may be a distance that is not the distance between the proteins in a disease-free cell and/or sample. The diagnostic methods of the invention can be used to detect the presence of a disorder associated with aberrant molecule (e.g. protein) conformation and/or abnormal distance between two or more proteins, as well as to assess the progression and/or regression of the disorder such as in response to treatment (e.g., chemotherapy, pharmaceutical, or radiation). According to this aspect of the invention, the method for diagnosing a disorder characterized by aberrant molecule conformation or distance between molecules: detecting in a first biological sample obtained from a subject, the conformation or distance, wherein a change in the conformation or distance compared to a control sample indicates that the subject has a disorder characterized by aberrant conformation or distance, respectively. It will be understood by one of ordinary skill in the art that a baseline molecule conformation or distance between two or more molecules can also be used in the assessment of disorders that are associated with change in molecule conformation and/or distance between two or more molecules.

As used herein, a “biological sample” or “sample” includes, but is not limited to: tissue, cells, or body fluid (e.g., blood). A fluid sample may include cells and fluid. The tissue and cells may be obtained from a subject or may be grown in culture (e.g. from a cell line). The tissue or cells may be obtained (e.g., from a tissue biopsy, aspiration, or fluid collection) using methods well known to those of ordinary skill in the related medical arts. A cell may be any type of cell including, but not limited to, neuronal cells. The methods of the invention may be used to monitor molecule conformation and/or the distance between two or more molecules in living cells or in dead (e.g. fixed) cells. In living cells, the monitoring can be done in vivo and without damaging the monitored cell. The dead (e.g. fixed) cells used in the invention may be affixed to a surface such as a glass slide or dish. As used herein the term “subject” means a mammal, including humans, non-human primates, dogs, cats, horses, pigs, cattle, sheep, and rodents, including but not limited to mice and rats.

As used herein, the terms “increase,” “decrease,” and “difference” preferably mean a significant increase, decrease, and difference respectively, e.g. statistically significant.

As used herein the term “control” means predetermined values, and also means baseline controls. Examples of controls include samples from control populations or control samples generated through manufacture to be baseline controls for experimental samples.

As used herein the term “control” includes positive and negative controls which may be a predetermined value that can take a variety of forms. The control(s) can be a single cut-off value, such as a median or mean, or can be established based upon comparative groups, such as in groups having normal conformation of a molecule of interest and/or normal distance between the two or more molecules of interest in cells and tissues and groups having abnormal conformation of a molecule of interest and/or normal distance between the two or more molecules of interest in cells and tissues. Another example of a comparative group is a group having a particular disease, condition and/or symptoms and a group without the disease, condition and/or symptoms. Another comparative group is a group with a family history of a particular disease and a group without such a family history of the particular disease.

The predetermined value of a control will depend upon the particular population selected. For example, an apparently healthy population (or cells or subjects) will have a different “normal” conformation of a molecule (e.g. protein) of interest and/or distance between two or more molecules of interest range than will a population which is known to have a condition characterized by aberrant molecule conformation and/or distance between the two or more molecules. Accordingly, the predetermined value selected may take into account the category in which an individual falls. Appropriate ranges and categories can be selected with no more than routine experimentation by those of ordinary skill in the art. Typically the control will be based on apparently healthy individuals in an appropriate age bracket.

The invention also includes methods to monitor the onset, progression, or regression of disorders associated with the abnormal conformation of a molecule (e.g. protein) of interest and/or abnormal distance between two or more molecules of interest in a subject. The methods may include, for example, obtaining cell or tissue samples at sequential times from a subject and assaying such samples for the conformation of a molecule (e.g. protein) of interest and/or distance between two or more molecules of interest using the monitoring methods of the invention. A subject may be suspected of having a disorder associated with the abnormal conformation of a molecule (e.g. protein) of interest and/or abnormal distance between two or more molecules of interest or may be believed not to have such a disorder and the sample can serve as a baseline level for comparison with subsequent cell or tissue samples from the subject.

Onset of a condition is the initiation of the physiological changes or characteristics associated with the condition in a subject. Such changes may be evidenced by physiological symptoms, or may be clinically asymptomatic. For example, the onset of a disorder associated with the abnormal conformation of a molecule (e.g. protein) of interest and/or abnormal distance between two or more molecules of interest may be followed by a period during which there may physiological characteristics in the subject, even though clinical symptoms may not be evident at that time. The progression of a condition follows onset and is the advancement of the physiological characteristics of the condition, which may or may not be marked by an increase in clinical symptoms. In contrast, the regression of a condition is a decrease in physiological characteristics of the condition, perhaps with a parallel reduction in symptoms, and may result from a treatment or may be a natural reversal in the condition. The methods of the invention can be use to monitor the onset, progression, or regression of a disorder associated with abnormal conformation of a molecule (e.g. protein) of interest and/or abnormal distance between two or more molecules of interest.

The invention also includes kits that include the donor and acceptor fluorophores of the invention. An example of a kit of the invention is a kit that provides components necessary to determine the conformation of a molecule (e.g. protein) of interest and/or distance between two or more molecules of interest in a cell or tissue sample. Components in such kits may include donor and acceptor fluorophores (e.g. linked to antibodies) and instructions for its use to assess molecules of interest. The kits of the invention can include instructions or other printed material on how to use the various components of the kits for diagnostic purposes. Additional materials may be included in any or all kits of the invention, and such materials may include, but are not limited to buffers, water, enzymes, tubes, control molecules, etc.

The invention further provides efficient methods of identifying pharmacological agents or lead compounds for pharmaceutical agents that affect the conformation of a molecule (e.g. protein) of interest and/or the distance between two or more molecules of interest. Generally, the screening methods involve use of the methods of the invention to assay for compounds that alter conformation of a molecule (e.g. protein) of interest and/or alter the distance between two or more molecules of interest.

Typically, a plurality of assays are run in parallel with different pharmaceutical compound concentrations to obtain a different response to the various concentrations. Typically, one of these concentrations serves as a negative control, i.e., at zero concentration of the pharmaceutical or at a concentration of the pharmaceutical compound below the limits of assay detection. Candidate agents encompass numerous chemical classes, although typically they are organic compounds. Preferably, the candidate pharmacological agents are small organic compounds, i.e., those having a molecular weight of more than 50 and less than about 2500, preferably less than about 1000 and, more preferably, less than about 500. Candidate agents comprise functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules are also contemplated.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc., which may be used to facilitate optimal activity of the assay. Such a reagent may also reduce non-specific or background interactions of the reaction components. Other reagents that improve the efficiency of the assay such as protease inhibitors, nuclease inhibitors, antimicrobial agents, and the like may also be used.

An exemplary assay to assess the conformation of a molecule (e.g. protein) of interest and/or the distance between two or more molecules of interest is described herein. In general, the mixture of the foregoing assay materials, including a donor and acceptor fluorophore, is incubated under conditions whereby, but for the presence of the candidate pharmacological agent, the level of fluorescence remains similar or equal to that of a control assay. The order of addition of components, incubation temperature, time of incubation, and other parameters of the assay may be readily determined. Such experimentation merely involves optimization of the assay parameters, not the fundamental composition of the assay. After incubation, the conformation of the molecule (e.g. protein) of interest and/or the distance between two or more molecules of interest can be detected and analyzed using the methods taught herein. (see Examples)

The invention will be more fully understood by reference to the following examples. These examples, however, are merely intended to illustrate the embodiments of the invention and are not to be construed to limit the scope of the invention.

### EXAMPLES

### Example 1

Introduction

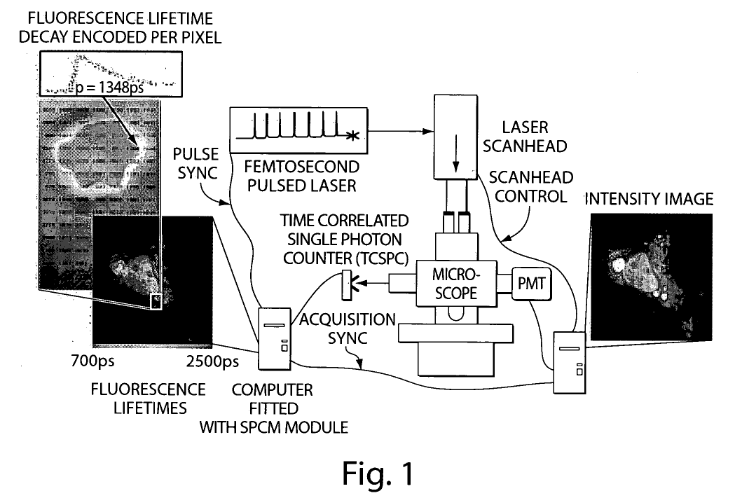

To examine amyloid β plaques, we used a commercial FLIM instrument in conjunction with a commercial laser scanning multiphoton microscope. The femtosecond pulsed near infrared laser is an ideal excitation source for time-domain fluorescence lifetime measurements. With synchronization from the x-y scanners, fluorescence lifetimes can be acquired on a pixel-by-pixel basis, with high spatial resolution. Multiexponential curve fits for each pixel results in 2-dimensional FRET measurements that allow the determination of both proximity of fluorescent FRET pairs, as well as the fraction of FRET pairs close enough for FRET to occur. Experiments are described that characterize this system, as well as commonly used reagents valuable for FRET determinations in biological systems. Constructs of CFP and YFP were generated to demonstrate FRET between this pair of GFP color variants. The lifetime characteristics of the FRET pair fluorescein: rhodamine; commonly used for immunohistochemistry were also examined. Finally, these fluorophores were used to demonstrate spatially resolved FRET with senile plaques obtained from transgenic mouse brain. Together these results demonstrate that FLIM allows sensitive measurements of protein-protein interactions on a spatial scale less than 10 nm using commercially available components. FIG. 1 provides a schematic diagram of a FLIM acquisition system.

We have investigated the characteristics of the commonly used FRET pair fluorescein and rhodamine using a commercially available multiphoton microscope and time-correlated single photon counting hardware and software for FLIM measurements. Our results demonstrated that FLIM measurements allow easy and quantifiable measures of FRET within biological tissue with high spatial resolution. These results lay the groundwork for quantitative FRET analysis in a variety of immunolabeled cells or tissue. We have also demonstrated that green fluorescent protein color variants are amenable to FRET measurements with FLIM using our system. After investigating some of the fundamental characteristics of the fluorescence lifetimes of fluorophores suitable for biological investigations, we have applied double immunofluorescence to tissue from transgenic mouse models of Alzheimer's disease. This disease is characterized by the progressive accumulation of senile plaques, comprised primarily of the amyloid-β peptide (Hyman, B. T. et al., J Neuropathol Exp Neurol. 56(10): 1095-1097, 1997; Markesbery, W. R., Neurobiology of Aging 18(4 Suppl): July-August, 1997). Amyloid-β forms β-sheet structures within subclasses of senile plaques, and the distribution of this morphological form of amyloid-β may occur on a multiple spatial scales, both between types of plaques and within individual plaques. We have demonstrated that double immunofluorescence with distinct anti-amyloid-β antibodies can lead to detectable differences in colocalization of epitopes within 10 nm, detectable by FRET using FLIM, possibly suggesting differences between intra- and inter-molecular FRET.

Methods

The FLIM Acquisition System.

A femtosecond pulsed Ti:Saphire laser (Tsunami, Spectra-Physics Lasers Inc., Mountain View, Calif.) is used for fluorescence excitation. This generates a pulse train at about 80 MHz. A commercial multiphoton microscope (Radiance 2000, Bio-Rad, Hercules, Calif.) is used for laser scanning, coupled to an upright microscope (BX50, Olympus America, Inc. Melville, N.Y.). Fluorescence imaging is achieved either with the multiphoton acquisition software (Lasersharp, Bio-Rad), or with the FLIM acquisition software (SPCImage, Becker&Hickl, Berlin, Germany). Fluorescence lifetimes are recorded using a high-speed photomultiplier (PMH-100, Becker&Hickl) and a fast time-correlated single photon counting acquisition board (SPC-830, Becker&Hickl), that allows high temporal resolution lifetime acquisition with high spatial resolution imaging. Laser pulse synchronization signals together with x-y scan synchronization signals allow assignment of collected photons resulting from each laser pulse on a pixel-by-pixel basis. Acquisition time depends on the sample, but generally requires several minutes of scanning. Fluorescence lifetimes are calculated from the raw data with single or multi-exponential curve fits through the 256 time bins within each pixel. A matrix is created with the curve fit data for each pixel, allowing color coding by lifetime, super-imposed over the intensity image.

FRET Detection of CFP and YFP with FLIM.

Genetic constructs were made that express CFP alone, YFP alone, or CFP-YFP as a fusion protein. These constructs were transfected into a clonal cell line (H4), and imaged in vitro with FLIM 24hrs after transfection. The cells were maintained in Hank's Balanced Salt Solution (Invitrogen, Carlsbad, Calif.) containing 10 mM HEPES at 22 degrees. Images were acquired using a 20× water immersion objective (Olympus, NA=0.95), for 3 min for each sample at 512×512 pixels, at a rate of 1 frame/sec. FLIM images were binned spatially to 128×128 pixels for these experiments. A 480/DF30 nm bandpass filter (Chroma Technology) with BG39 glass to block excitation wavelengths was used for image acquisition.

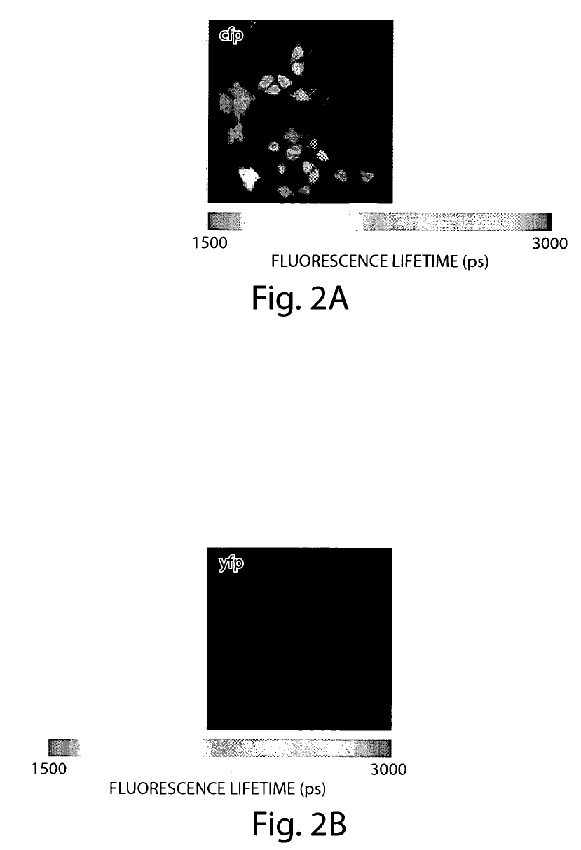

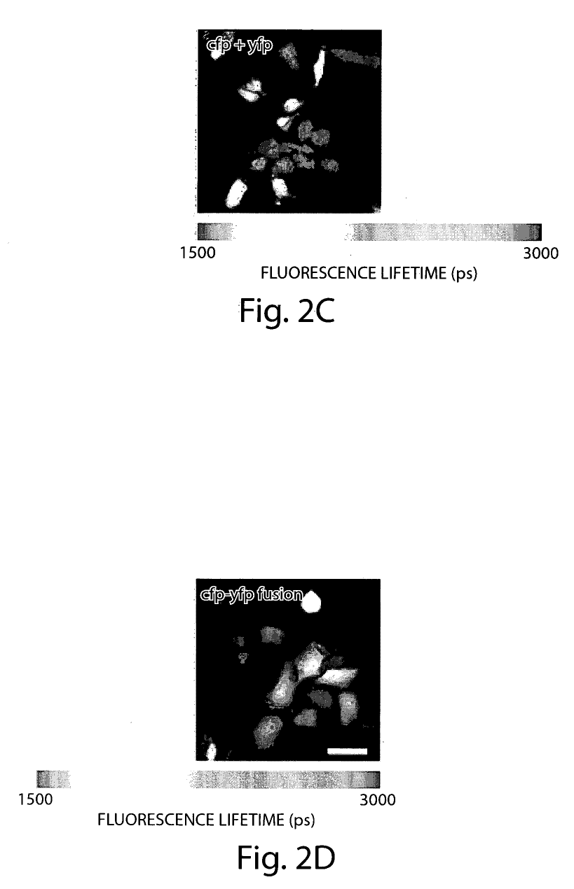

Cells expressing CFP alone had a single lifetime component of 2.9±0.1 ns (FIG. 2A). The images were pseuodocolored according to fluorescence lifetimes from 1.5 ns (blue) to 3.0 ns (red). YFP (FIG. 2B) was not excited with multiphoton excitation at 740 nm. CFP contransfected with YFP (FIG. 2C) yielded a fluorescence lifetime indistinguishable from CFP alone-transfected cells, 2.9±0.1 ns, indicating that there was no FRET between these individual proteins. However, when CFP was fused to YFP in the same construct, the fluorophores were forced into close proximity, and the lifetime of CFP was reduced in these cells to 1.9±0.1 ns, (FIG. 2D) indicative of FRET.

Determination of pH Dependence of Fluorescein and Fluorescein-labeled IgG Antibody.

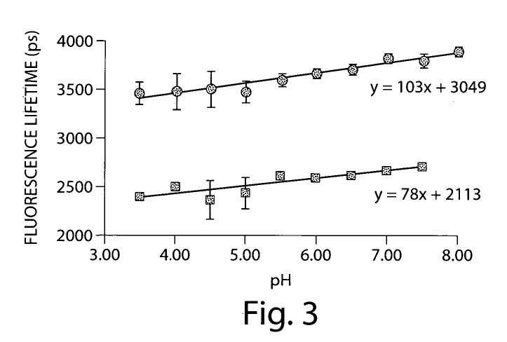

Samples of either free fluorescein or fluorescein-labeled antibody (10d5, Elan Pharmaceuticals), were prepared in TBS buffer supplemented with citrate, at varying concentrations of H+. Samples were loaded into glass capillary tubes (Borosilicate, 1 mm diameter, WPI), and placed on the stage of the multiphoton microscope. Each sample was scanned for 3 minutes while acquiring fluorescence lifetimes using a 20× water immersion objective (Olympus, NA=0.45). FLIM images were processed for lifetimes, and summary data for each sample were obtained by averaging the lifetimes within a large region of interest in the spatially uniform field. Measurements were performed in triplicate for each sample. The free fluorescein solutions had longer lifetimes than the fluorescein-labeled antibody, (FIG. 3) and each show a weak dependence on pH, with the lifetimes higher at elevated pH.

Lifetime Dependence of Labeled Streptavidin Complex.

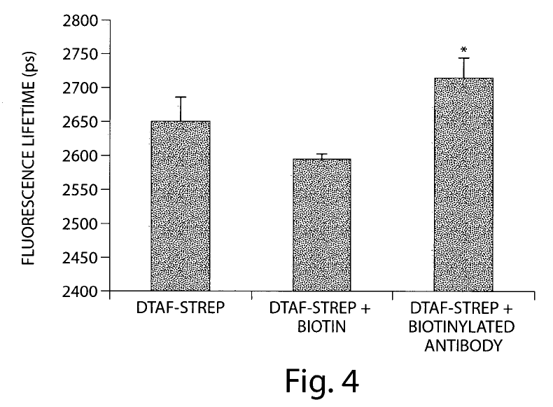

Solutions of DTAFstreptavidin (Jackson ImmunoResearch Laboratories, West Grove, Pa.) were placed in glass capillary tubes at 0.1 mg/ml in PBS. Fluorescence lifetime images were acquired for 3 min, using 800 nm excitation, and a green emission filter (515df30). Average values were determined from within each sample. The streptavidin-antibody solution resulted in aggregates that precipitated, (FIG. 4) but still allowed lifetime measurements. Fluorescein-labeled streptavidin with free biotin led to a slight, but insignificant decrease in fluorescence lifetime. In the presence of biotinylated antibody, however, the lifetimes are modestly elevated, suggesting a de-quenching of the fluorophore (p<0.05, Student's t-test).

Determination of a Calibration Curve for Mixed Populations of FRET and Non-FRET Pairs.

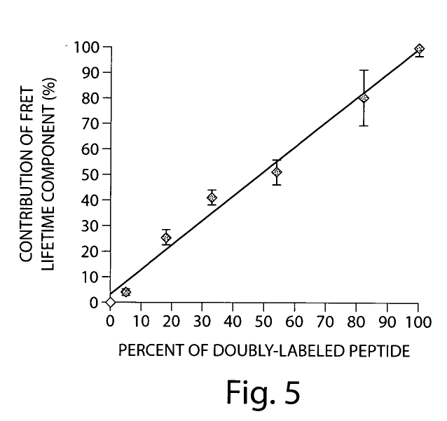

An 8 amino acid peptide was synthesized and labeled with fluorophores. The peptide sequence was GKVQIVYK (SEQ ID NO:1), which is homologous to a region of the human tau protein (von Bergen, M. et al., Proc Natl Acad Sci U S A. 97(10): 5129-5134, 2000). Fluorescein was conjugated to the N-terminal residue, and rhodamine was attached to the C-terminal amino acid. The doubly labeled peptide was a positive control for FRET, since the two fluorophores are separated by only 8 amino acids, well within the range for FRET. Singly labeled peptides were also made with either fluorescein or rhodamine on the appropriate peptide terminus. FLIM was used to characterize the three peptides in solution at 0.1 mg/ml in EtOH/H₂O at 70%/30% using a green bandpass filter (515/df30) to select for the fluorescein (donor) fluorescence. No signal was obtained from the rhodamine alone labeled peptide when exciting at 800 nm for multiphoton fluorescence. The fluorescein alone sample resulted in a homogenous monoexponential curve fit with a mean lifetime of 3.2±0.1 ns. The doubly labeled peptide had two lifetime components, at 3.2±0.1 ns, and at 1.4±0.2 ns. The amplitude of each of these lifetimes was approximately 50%, indicating that only half of the doubly labeled peptide was exhibiting FRET, suggesting that the peptide was a 50:50 mixture of peptide that was doubly labeled and peptide labeled with fluorescein alone. Mixtures of singly labeled and doubly labeled peptides were made at varying ratios, corrected for the measured concentration of doubly labeled peptide. These samples were placed into glass capillary tubes and imaged using FLIM. Double exponential curve fits were performed on the samples, fixing the fluorescence lifetimes at 1.4 and 3.2 ns, and calculating the amplitude corresponding to these lifetimes, which represent the fraction of each of these lifetimes within each sample. The calibration curve (FIG. 5) plots the calculated amplitude of the fast lifetime component as a function of the fraction of doubly labeled peptide in the sample mixtures. The results demonstrate that the curve-fitting routine reliably estimates the fraction of FRETing to non-FRETing molecules within a sample, on a pixel-by-pixel basis. This is a significant advantage of using time-domain FLIM for FRET measurements as compared with other FRET determinations, which cannot discriminate between weakly associating fluorophore pairs, or a mixed population of strongly associating and non-associating pairs.

Determination of the Morphology of Senile Plaques Like Those Found in Alzheimer's Disease.

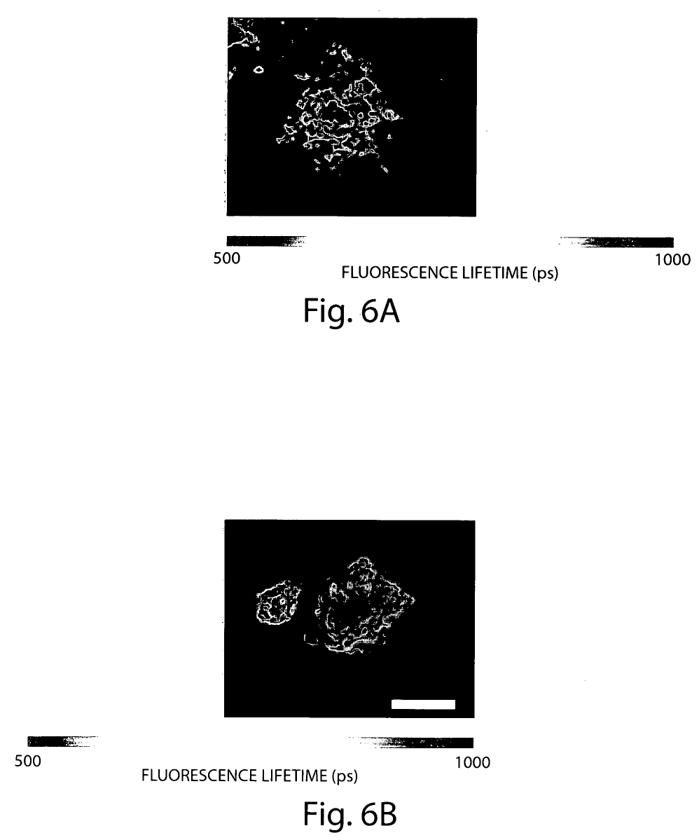

Cryostat sections from 18-month-old Tg2576 mouse brains were processed for immunohistochemistry, as previously described. Directly labeled antibodies against the amyloid-β peptide were used. FITC-labeled 10d5 was used alone in some tissue sections, and both FITC-10d5 and rhodamine-labeled 3d6 antibody were used together in other sections. These antibodies both recognize epitopes at or near the N-terminus of amyloid-β, but do not compete with each other for binding. FLIM acquisition was performed using a 515df30 23 interference filter, and a 20× water immersion objective (Olympus, NA=0.45). Excitation was at 800 nm, and fluorescence was acquired for 3 min per sample. 3 tissue sections from 3 different mice were used for each sample, acquiring images of approximately 18 plaques for each case. As shown in FIG. 6A, the fluorescence lifetime of FITC-10d5 immunolabeling was uniform across the plaques, with a mean lifetime of 2.6±0.1 ns. FIG. 6B illustrates a representative example of a plaque that was doubly labeled with FITC-10d5 and rhodamine-3d6. In this example, FRET was observed throughout the plaque, as evidenced by the reduction in the fluorescence lifetime of fluorescein to 0.9±0.1 ns. Interestingly, the lifetimes were no longer uniform across the plaque, but show spatially distinct differences from the core of the plaque to the periphery. The core exhibited longer lifetimes, and less FRET than the fringes. Our results indicate that senile plaques like those found in Alzheimer's disease are morphologically nonuniform.

Results

We have used commercial multiphoton microscopes for in vivo imaging of senile plaques in transgenic mouse models of Alzheimer's disease(Bacskai, B. J., et al., Nature Medicine 7(3): 369-372, 2001; Christie, R. H. et al., J Neurosci. 21(3): 858-864, 2001; Bacskai, B. J. et al., J Neurosci. 22(18): 7873-7878, 2002; Bacskai, B. J. et al., J Cereb Blood Flow Metab. 22(9): 1035-1041,2002). Multiphoton microscopy depends on pulsed near infrared lasers with high peak power, which was achieved when the pulses are ˜100 fs, as with Ti:Sapphire lasers (Tsunami, 10 W Millenia Xs pump laser, Spectra Physics). These lasers are not inexpensive, but provided a pulsed excitation source for time-domain FLIM, with a repetition rate at about 80 MHz. At this rate, a new pulse occurred every 12.5 ns or so, which allowed a sampling rate that was above the Nyquist frequency for most fluorescence lifetime applications. The laser was coupled to a commercial multiphoton microscope (Radiance 2000, Bio-Rad) that allowed several scanning speeds and multiple image sizes. Synchronization signals from the Biorad scanhead were fed directly to the high-speed time correlated single photon counting (TCSPC) acquisition hardware (SPC-830, Becker&Hickl). This allowed pixel-by-pixel registration of the accumulated photons with the laser scanning. FIG. 1 shows a schematic of the optical setup. The FLIM acquisition board allowed up to 4096×4096 pixels with reduced temporal resolution, or up to 2048 time bins at lower spatial resolution for the most accurate lifetime determinations. We routinely used 128×128 pixels at 256 time bins per pixel (˜40 ps per time bin) as a compromise between short acquisition times, good spatial resolution, and high temporal resolution of the fluorescence decays.

The choice of detectors was important, since it was desirable to have both high sensitivity, and very fast response times. We used a fast photomultiplier tube, with a FWHM response time of about 150 ps (PMH-100, Becker&Hickl). This detector was fast enough to resolve lifetimes shortened by FRET, but the detector is also rugged, reliable, inexpensive, and suitable for a multi-user facility with both experienced and inexperienced operators. The data analysis software (SPCImage, Becker&Hickl) allowed multi-exponential curve fitting of the acquired data on a pixel-by-pixel basis using a weighted least-squares numerical approach (O'Connor, D. V. et al., London: Academic Press, 1984). The sum of all time bins was equivalent to the intensity image, and this was displayed to an image pseudocolored according to the curve fit results. A matrix was created with the curve-fit data for each pixel, and a pseudocolor range could be defined for the exponential decay time results. The images could be color-coded to display the distribution of any one of three exponential decay times, or the mean decay per pixel. Therefore, each image could be easily displayed in a meaningful way to compare lifetimes within or between other images.

In order to characterize this system for meaningful FRET results, we began with genetic constructs expressing color variants of green fluorescent protein (GFP). We made constructs based on the established FRET pair CFP-YFP (Chan, F. K. et al., Cytometry 44(4): 361-368, 2001; Harpur, A. G. et al., Nat Biotechnol. 19(2): 167-169, 2001; Pepperkok, R. et al., Curr Biol. 9(5): 269-272, 1999). Expression vectors for each of these fluorophores were made, as well as a fusion construct linking CFP to YFP for maximal FRET. Using these tools transfected into live cells, we imaged the lifetimes of CFP alone, YFP alone, and the fusion of CFP-YFP, as shown in FIG. 2. Fluorescence emission was collected using a 480 nm bandpass filter and BG39 glass for rejection of the excitation source, which was tuned to 740 nm. This wavelength was chosen because it excites CFP without exciting YFP, minimizing crosstalk. The results show that CFP alone exhibited a single fluorescence decay of 2.9±0.1 ns that was homogeneously distributed throughout each cell. When fused to YFP, however, the single fluorescence lifetime of CFP was shortened to 1.8±0.1 ns. These values were obtained by averaging the single exponential curve-fit results within areas of interest within groups of cells for each condition. CFP cotransfected with YFP leads to a lifetime indistinguishable from that of CFP alone, since there is no FRET, and YFP alone is not detected with this choice of excitation wavelength and emission filter. The absence of signal in the YFP alone-transfected cells with these imaging conditions suggests that autofluorescence contributes a negligible signal in the bright, CFP transfected cells. These results demonstrated that FRET between CFP and YFP can be easily detected using FLIM, permitting experiments with genetically encoded functional markers based on FRET between this pair of fluorophores (Ting, A. Y. et al., Proc Natl Acad Sci USA 98(26): 15003-15008, 2001; Miyawaki, A. et al., Nature 388(6645): 882-887, 1997).

We previously determined FRET between pairs of antibodies targeted with appropriately labeled antibodies (McLean, P. J. et al., J Biol Chem. 275(12): 8812-8816, 2000; Knowles, R. B. et al., J Neuropathol Exp Neurol. 58(10): 1090-1098, 1999; Kinoshita, A. et al., J Neurosci. 21(21): 8354-8361, 2001; Sharma, N. et al., Acta Neuropathol (Berl) 102(4): 329-334, 2001; Sharma, N. et al., Am J Pathol. 159(1): 339-344, 2001). We sought to both confirm the validity of FRET measurements with FLIM, and to understand better the potential conflicts from environmental factors, such as concentration dependence, pH, and conjugation to proteins on the fluorescence lifetimes. FIG. 3 shows the pH dependence of the lifetime of fluorescein in solution, both free, and coupled to an IgG antibody. Fluorescent samples were placed in glass capillary tubes, and imaged with a 20× water immersion objective (Olympus). The concentration of fluorophore in each sample was 10 μM. We found no differences in measured lifetimes from concentrations that were barely detectable up to about 1 mM fluorescein, where homo-FRET or inner filtering effects made measurements noticeably complex. Photons were acquired for 3 minutes per sample, increasing the laser intensity as necessary for low pH samples, since the quantum yield of fluorescein drops significantly at these levels. Care was taken not to exceed the count rate of the detector, which was 2×10⁶ photons/sec. The resulting images accrued about 1×10⁴ photons per pixel, distributed over the 10 ns lifetime acquisition range. Average values from the curve-fit results were determined from approximately 6000 pixels in a rectangular area of interest for each sample. The results demonstrated that the lifetime of fluorescein at pH 7.4 was shortened from ˜3.9 ns to ˜2.6 ns when covalently labeled to an antibody, and that there was a weak linear decrease in lifetime with decreasing pH. Both free and labeled fluorescein exhibited a similar dependence on pH. These results indicate that the decrease in lifetime when the antibody is labeled is important for interpreting FRET data, since a comparison cannot be made with established values of the lifetime of free fluorescein, but must be made with measured values of each labeled antibody. The pH dependence of the lifetime of fluorescein supports previously reported values describing different prototropic forms of fluorescein (Klonis, N. and W. H. Sawyer, Journal of Fluorescence 6(3): 147-157, 1996). In our samples, however, we did not observe complex exponential decays of fluorescein fluorescence at any pH, despite the probable existence of multiple lifetimes in some of the samples. This may reflect an inability of our system to differentiate lifetimes that are close to each other. The use of faster photon counting detectors, like a microchannel plate detector and/or increased temporal resolution of the TCSPC acquisition may permit resolution of mixtures of fluorophores with similar lifetimes.

A popular technique for targeting fluorophores to biologically relevant molecules relies on the highly specific and sensitive binding of biotin and streptavidin. With this technique, biotinylated antibodies are used to target the epitopes, and fluorescently labeled streptavidin conjugates are used to target the biotin. FIG. 4 demonstrates the effect on the fluorescence lifetime of fluorescein when bound to streptavidin in the presence of either free biotin, or biotinylated antibody. Samples buffered at pH 7.4 were placed in glass capillary tubes and lifetime images were acquired for 3 min per sample. The lifetimes for each sample were determined by averaging the curve-fit results in rectangular areas of interest, comprising at least 5000 pixels. Samples were prepared and imaged in triplicate. The lifetime of fluorescein streptavidin (DTAF-streptavidin, Jacskon ImmunoResearch Laboratories, Inc.) is 2.6±0.03 ns, and is unaffected by the presence of free biotin 2.6±0.01 ns, but increased slightly by the presence of biotinylated antibody (goat anti-mouse, Jackson ImmunoResearch Laboratories, Inc., 2.7±0.03 ns, p<0.05, Student's t-test). Within each sample, the pixel-topixel noise was less than 50 ps, allowing separation of the two lifetime values, but probably only within bright samples, and not biological specimens. This result indicates that the streptavidin-biotin technique for immunofluorescence is slightly, but significantly sensitive to the direct labeling of the biotinylated antibody, and is amenable to FRET determinations, similar to the results obtained with fluorescein labeled antibodies.

In order to show FRET between fluorescein and rhodamine in a controlled manner, we made peptides with either single or double labels of fluorescein and rhodamine. An 8 amino acid peptide was labeled either singly, at the N-terminus, or doubly at each terminus. In the absence of any secondary structure, the N and C terminus of an 8-mer is well within the Forster's distance for FRET. In fact, the fluorescence lifetime of fluorescein decreases from 3.2±0.1 ns in the singly labeled peptide to 1.6±0.05 ns in the doubly labeled peptide. This result indicates that our FLIM setup is easily able to measure FRET between this fluorescein and rhodamine. One of the advantages of using FLIM to measure FRET, however, particularly with time-domain measurements, is that it is possible to not only measure the degree of FRET on a pixel-by-pixel basis, but also the proportion of FRETing molecules within each pixel. This is achieved since the curve-fitting procedure results in both a lifetime measure and an amplitude measure, such that with a multiexponential fit, the fraction of FRETing molecules is deconvolved with the degree of FRET. To test the sensitivity of this measure with our system, we mixed, in varying ratios, known concentrations of singly and doubly labeled peptides to generate a calibration curve, as shown in FIG. 5. Each sample had two FRETing populations, but at different proportions. By fixing the lifetimes during curve fitting at each of the measured lifetimes of the 0% and 100% samples, the amplitudes of the exponents became the dependent variables. The amplitudes provided a measure of the proportion of fluorescence lifetimes contributing to each decay. The mixtures exhibited a linear response in the amplitude of the FRET-derived fluorescence lifetime, indicating that our experimental technique was capable of resolving both the degree of FRET and the proportion of FRET in a spatially resolved way. This result demonstrates that two lifetimes in a mixed population could be distinguished, and assigned a relative amplitude that was proportional to their relative concentration. While we used a mixture of FRETing and non-FRETing fluorescein in this example, this approach can be used to resolve mixtures of fluorophores, even spectrally similar fluorophores, as long as their lifetimes are sufficiently separable.

After establishing some baseline parameters for FRET determinations, we evaluated our FLIM system in histological sections. We applied directly labeled antibodies to tissue from mouse brain. The brains were from 20-mo-old Tg2576 mice, which overexpress mutant human amyloid precursor protein (APP), leading to the progressive development of senile plaques, similar to those found in Alzheimer's disease tissue (Hsiao, K. et al., Science 274(5284): 99-102, 1996). Senile plaques occur with various morphologies, including compact or dense-core plaques, as well as diffuse aggregates, all resulting from deposition of the amyloid-β peptide in the brain (Yen, S. H. et al., Neurobiology of Aging 16(3): 381-387, 1995; Dickson, D. W. Neurobiology of Aging 18(4 Suppl): July-August, 1997). It is known that the amyloid-β peptides deposit in β-pleated sheet conformation in dense-core plaques, allowing easy detection with histological stains that recognize β-sheet structures, and not necessarily amyloid-β specifically (Kelenyi, G., Acta Neuropathol (Berl) 7(4): 336-348, 1967). Antibodies, however, recognize distinct epitopes of the amyloid-β peptide, and can be used to target both dense-core and diffuse deposits. We used a fluorescein labeled antibody (10d5, Elan Pharmaceuticals, (Hyman, B. T. et al., J Neuropathol Exp Neurol. 51(1): 76-83, 1992)) that recognizes amino acids 3-6 of amyloid-β and labels all morphological types of senile plaques. FLIM acquisition of antibody-labeled plaques revealed homogenous lifetimes within and between plaques (FIG. 6). Double immunofluorescent labeling, however, of the same plaques with another anti-amyloid-β antibody (3d6, recognizing amino acids 1-5, Elan Pharmaceuticals, (Hyman, B. T. et al., J Neuropathol Exp Neurol. 51(1): 76-83, 1992)) labeled with rhodamine revealed a different pattern of fluorescence lifetimes. First, there was detectable FRET between the antibodies throughout the plaques. The fluorescence lifetimes of fluorescein decreased from 2.6±0.1 ns in the fluorescein alone tissue, to 1.9±0.1 ns in the doubly labeled tissue. We have previously shown that these two antibodies co-localize with each other on amyloid-β deposits, despite the nearly overlapping epitopes near the N-terminus of amyloid-β (Bacskai, B. J. et al., J Neurosci. 22(18): 7873-7878, 2002). Our FLIM results demonstrate that the antibodies are, indeed, extremely close to each other, within 10 nm. This distance is closer than can be determined by colocalization studies. Surprisingly, we also observed spatially distinct profiles of FRET within individual plaques. In dense-core plaques, there was a higher degree of FRET in the periphery of the plaque versus the core. This was demonstrated most effectively in the pseuodocolored image of FIG. 6b. This result indicates that the amyloid-β peptide has a different 3-dimensional conformation in the core of a plaque as compared to the periphery or to diffuse plaques. Importantly, this is not due to packing density (since FLIM is concentration independent (Elangovan, M. et al., J Microsc. 205(Pt 1): 3-14, 2002; Lakowicz, J. R. et al., Anal Biochem. 202(2): 316-330, 1992)) but implies a difference in the relative conformation of the two epitopes to one another. We expect inter-molecular FRET to be less close (hence longer lifetimes) compared to intra-molecular FRET, and this therefore suggests that the core of a plaque may favor a β-pleated sheet packing orientation that favors intermolecular interactions between N-termini of amyloid-β peptides. This result is pursued further with a range of antibodies with discrete epitopes along the sequence of amyloid-β.

Discussion

We have developed a time-domain FLIM system using commercially available components. By coupling a high-speed TCSPC acquisition board with a laser scanning multiphoton microscope, we were able to obtain high spatial resolution images with high temporal resolution lifetimes on a pixel-by-pixel basis. Using this system, we characterized the fluorescence lifetime of fluorescein conjugated to monoclonal antibodies and streptavidin reagents, commonly used for immunohistochemistry. We also demonstrated FRET between cells transfected with green fluorescent protein color variants, further establishing their usefulness for addressing both static and dynamic determinations of protein interactions (Wouters, F. S. et al., Curr Biol. 9(19): 1127-1130, 1999; Harpur, A. G. et al., Nat Biotechnol. 19(2): 167-169, 2001; van Kuppeveld, F. J. et al., J Virol. 76(18): 9446-9456, 2002; Ng, T. et al., Science. 283(5410): 2085-2089, 1999). High resolution FLIM permits detection of FRET in doubly labeled cells and tissue, permitting colocalization of protein pairs with resolutions less than 10 nm. As opposed to frequency domain FLIM, this system permits determinations of both degree of FRET as well as the proportion of molecules within a volume exhibiting FRET.

We also describe the implementation of this system for FRET determinations in biological tissue from a transgenic mouse model of Alzheimer's disease. This mouse develops senile plaques similar to those found in the human disease, and characterization of these neuropathological lesions in vitro or in vivo will aid in our understanding of the disease. Our results indicate that individual senile plaques exhibit two morphological conformations of amyloid-β peptide, detectable with FLIM. This may provide a clue as to the initial formation of senile plaques, or to the maturation of a plaque over time. Further experiments with a range of antibodies recognizing specific epitopes within the amyloid-β peptide, and transgenic mice at an age when plaques are just beginning to appear may help elucidate these issues.

FLIM is a powerful spectroscopic technique for analyzing FRET interactions on a spatial scale of less than 10 nm. With the recent availability of commercially available electronics for high-speed detection of fluorescence lifetimes, coupled with the recent popularity of laser scanning multiphoton microscopes, this technique should see more widespread use for ultra-high resolution analysis of protein-protein interactions in complex biological tissue.

### Example 2

Introduction

γ-Secretase cleavage is the final enzymatic step generating β-Amyloid (Aβ) via intramembranous cleavage of the Amyloid Precursor Protein (APP). Presenilin, initially identified as a gene in which mutations account for the vast majority of early onset autosomal dominant Alzheimer's disease, is a major component of γ-secretase. Enzymatic activity also depends on Nicastrin, Aph-1, and Pen-2. We developed a model in which γ-secretase components assemble, interact with substrates initially at a docking site, then cleave and release substrates. To test this model, we developed a novel morphological technique based on advanced fluorescence microscopy methods, Fluorescence Lifetime Imaging Microscopy (FLIM). FLIM allows us to examine protein-protein proximity in intact cells. We have shown that, although the strongest colocalization of APP and PS1 is in the perinuclear area, the strongest interactions detected by FLIM are at or near the cell surface. We also found that APP-PS1 interactions occur even when γ-secretase inhibitors or dominant negative PS-1 mutations are used to block γ-secretase activity. Finally, using Nicastrin RNAi, we have demonstrated that Nicastrin is critical for APP association with PS1. Our results suggest that there is a non-catalytic docking site closely associated with PS1/γ-secretase.

Methods

Cell Lines and Drug Treatments.

Chinese hamster ovary (CHO) cells stably transfected with APP770 and either wild type PS1 (wt PS1) or Aspartate mutant PS1 (D257A PS1) were grown in OPTI-MEM with 5% fetal bovine serum and appropriate antibiotics (Puromycin or Zeocin, respectively, (Berezovska et al., 2000)). To inhibit γ-secretase activity (to modulate or block the active site) cells were incubated for four hours with either a small dipeptide analogue γ-secretase inhibitor, DAPT (500 nM, (Dovey, H. F. et al. J Neurochem 76: 173-181, 2001), or a (hydroxyethyl)urea peptidomimetic WPE-III-31C (1 μM, (Esler, W. P. et al., Proc Natl Acad Sci USA 99: 2720-2725, 2002)) prior to immunostaining. HEK 293 cells were utilized for the RNAi experiments.

Immunocytochemistry/Antibodies.

Cells were fixed and immunostained 24 hrs posttransfection as described previously (Jack, C. et al., Brain Res Mol Brain Res 87: 166-174, 2001). We used a polyclonal C8 antibody (Selkoe, D. et al., Proc Natl Acad Sci USA 85: 7341-7345, 1988) to detect the C-terminus of APP. A monoclonal antibody to the loop region of PS1 (Chemicon, Temecula, Calif.) was used to identify the γ-secretase complex. PS1 N-terminal X81 antibody was a gift from D. Selkoe (Brigham and Women's Hospital, Boston, Mass.); PS1 C-terminal antibody was purchased from R&D Systems, Inc., Minneapolis, Minn.). For FRET/FLIM studies we chose Cy3-labeled goat anti-rabbit as the acceptor fluorophore, and FITC-labeled goat anti-mouse as the donor fluorophore.

FLIM and FRET Assays

Fluorescence Lifetime Imaging Microscopy (FLIM) relies on the observation that fluorescence lifetimes (the time of fluorophore emission after brief excitation, measured in picoseconds) are shorter in the presence of a FRET acceptor. A mode-locked Ti-sapphire laser (Spectra Physics) sends a femtosecond pulse every 12 nsec to excite the fluorophore. Images were acquired using a BioRad Radiance 2000 multiphoton microscope. We used a high-speed Hamamatsu detector and hardware/software from Becker and Hickl (Berlin, Germany) to measure fluorescence lifetimes on a pixel-by-pixel basis. Donor fluorophore fluorescein isothiocyanate (FITC) lifetimes were fit to two exponential decay curves to calculate the fraction of fluorophores within each pixel that either interact, or do not interact, with an acceptor. These lifetimes were then mapped by pseudo-color on a pixel-by-pixel basis over the entire image.

Validation of the FLIM Assay.

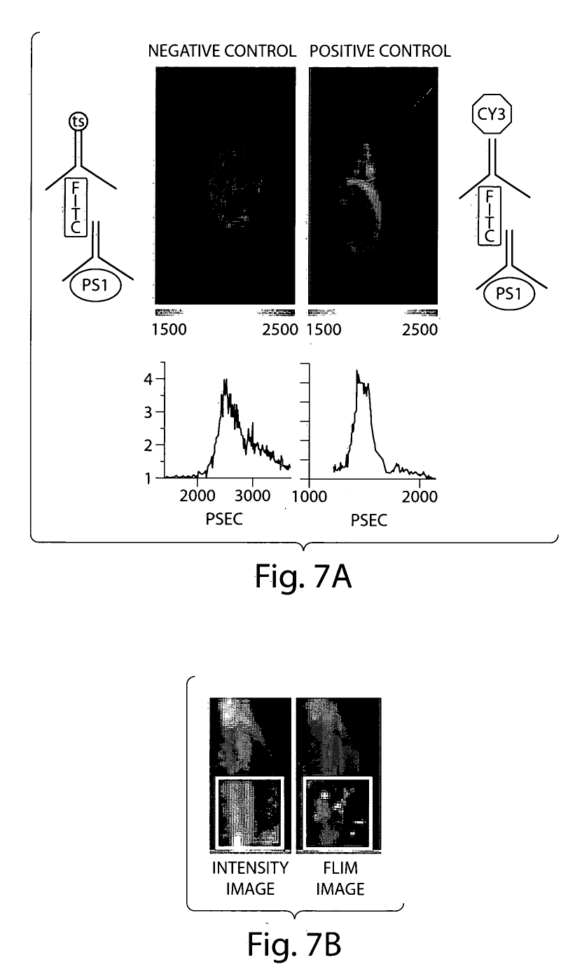

The following controls were used to establish and validate the FLIM assay: (1) As a negative control, FITC lifetime was measured in the absence of the acceptor. The cells were double stained with primary antibodies, followed by secondary antibodies including FITC-labeled and non-fluorescent labeled antibodies (no acceptor fluorophore). (2) As a positive control, FITC lifetime was measured in the presence of an acceptor. For this the cells were immunostained for presenilin using a standard monoclonal anti-loop domain antibody. This antibody was visualized with a FITC labeled goat-anti-mouse antibody, followed by reacting with a donkey-anti-goat antibody labeled with Cy-3 to bring FITC and CY-3 into a close proximity (see FIG. 7A) (Kinoshita, A. et al., J Neurosci 21: 8354-8361, 2001). (3) Next we used our positive control (Cy3-anti-FITC) to select an area within the cell (one-half of the cell); where the Cy-3 acceptor is destroyed by photobleaching by scanning the area for 30 sec with 568 nm light at 100% laser power. The resulting intensity image shows an increase in FITC intensity because the FITC is “dequenched” in the area where the CY-3 was photobleached (FIG. 7B). By contrast, the FITC fluorescence lifetime (FLIM image) within the area where the acceptor was photobleached reverts to a longer lifetime (color-coded as blue), identical to the lifetime of FITC in the absence of an acceptor (as in the negative control, FIG. 7A). This control shows close correlation between the FLIM and photobleaching FRET assays. (4) Another negative control we used was counterstaining of the cells with an antibody predicted to colocalize with, but not interact with PS1. PS1 is expressed in the ER. Double immunostaining shows that PS1 co-localizes with an ER resident protein, BiP. However, despite colocalization, the FITC (PS1) lifetime is the same as in the negative control, suggesting that BiP (Cy3 labeled) does not act as an acceptor. This control demonstrates that a change in the fluorescence lifetime, indicating FRET, is a more sensitive measure of proximity than simple co-localization.

Photobleach Dequenching

Fluorescence Resonance Energy Transfer (FRET) measurements were made using a BioRad1024 confocal microscope mounted on a Nikon Eclipse TE300 inverted Microscope. A krypton-argon laser was used to excite FITC and Cy3 (excitation wavelengths at 488 and 568 nm, respectively). FRET was measured using a method developed for laser scanning confocal microscopy (McLean, P. J. et al., J Biol Chem 275: 8812-8816, 2000; Kinoshita, A. et al., J Neurosci 21: 8354-8361, 2001). The amount of FRET was calculated as a percent increase in donor (FITC) fluorescence intensity after photobleaching the acceptor (Cy3) in a small part of the cell (˜8×8 μm). This ratio was then normalized to the percent change in FITC fluorescence intensity in a non-bleached area of the cell. The percent change in fluorescence intensity was compared to the null hypothesis value of 1.0 by one-group t-tests.

Nicastrin RNAi

We used a 21-mer double stranded RNAi (Dharmacon Research, Inc., Boulder, Colo.) directed against Nicastrin, Nct (Edbauer, D. et al., Proc Natl Acad Sci USA 99: 8666-8671, 2002). Cells grown in 60-mm dishes were transfected with 20 μl of 20 μM Nct RNAi using Effectene reagents (Qiagen, Valencia, Calif.) according to the manufacturer's instructions. 72 hours post-transfection the cells were harvested, lysed in a buffer containing 1% Triton-X100, and resolved by electrophoresis on a 10-20% Tris-Glycine gel (Invitrogen, Carlsbad, Calif.). HEK 293 cells showed the strongest suppression of Nicastrin expression (H4 and CHO cells were also tested), and were used for the FLIM analysis.

Results

To analyze the proximity between different molecules in intact cells we used two complementary assays, a photobleach dequenching FRET assay we have utilized for confocal microscopy (Kinoshita, A. et al., J Neurosci 21: 8354-8361, 2001), and a FLIM technique performed on a multiphoton microscope.

FLIM Reveals a Close Association of PS1 and APP in Distal Subcellular Compartments.

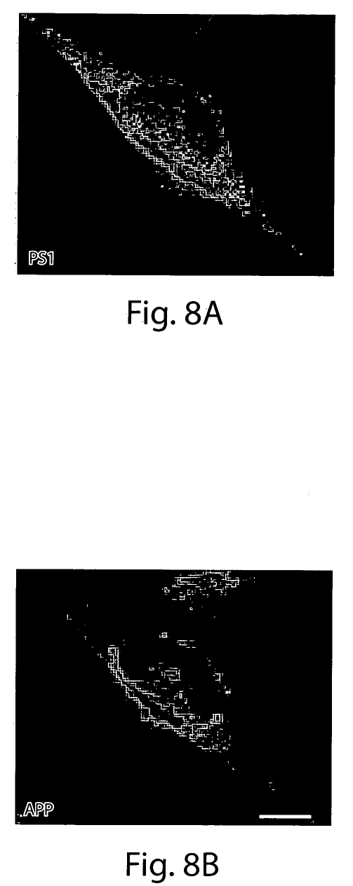

Confocal microscopic analysis of cells double-stained with anti-PS1 loop (Chemicon Inc., Pittsburg, Pa.) and C-terminal APP (C8, (Selkoe, D. et al., Proc Natl Acad Sci USA 85: 7341-7345, 1988)) antibodies revealed extensive perinuclear co-localization of APP and PS1 as well as lower signal in distal cell compartments (FIG. 8). This method shows subcellular compartment colocalization but not necessarily a close intermolecular interaction. We used FLIM to determine where in the cell APP and PS1 could be found in the closest proximity by analyzing the lifetime of the donor fluorophore.

Fluorescence lifetime is influenced by the surrounding microenvironment, and is shortened in the immediate vicinity of a FRET acceptor fluorophore. The degree of lifetime shortening is inherently a quantitative measure of proximity and changes in this quantity reflect alterations in conformation which can be displayed with very high spatial resolution in a pseudo-color-coded image. The fluorescence lifetime is displayed as a color: if molecules are closer together, the donor fluorescence lifetime will be shorter, and the color will be closer to red. We measured changes in the lifetime of FITC (the donor fluorophore) under different experimental conditions. Our negative control (in the absence of an acceptor fluorophore) showed that the lifetime of FITC (conjugated to IgG, hereafter referred to simply as FITC) alone is ˜2600±110 psec, whereas in the positive control (Cy3-anti-FITC), the FITC lifetime shortens to 1400±59 psec (FIG. 7).

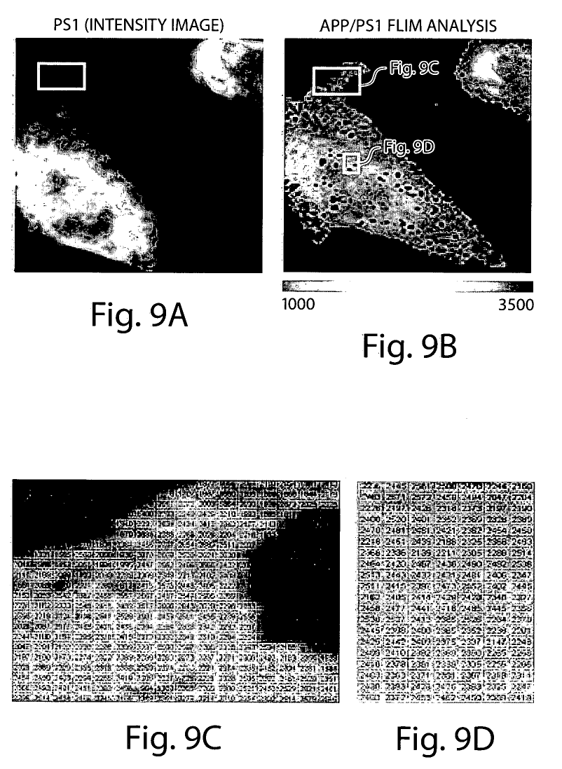

In each experiment PS1 loop was stained with FITC and APP (including the APP C-terminal fragments, C83 and C99) was stained with Cy3. While strongest co-localization occurred in the Golgi and ER as seen by confocal microscopy (FIG. 8), FLIM analysis showed that APP and PS1 did not interact closely in these compartments (the green-blue staining in the FLIM image, FIG. 9). Instead, PS1 and APP came into closest spatial proximity (the red pixels in FIG. 9) primarily in more distal compartments, near the cell surface. To analyze the fluorescence lifetime in each pixel of the imaged cell, a pseudo-colored and enlarged FLIM image (each individual pixel ˜0.04 μm2) was superimposed onto a look-up table with the average lifetimes for each pixel (FIG. 9B). We found that the average fluorescence lifetime was about 2118±190 psec on the periphery of the cell and 2402±49 psec in the perinuclear (ER/Golgi) compartments (P<0.05, compared to 2600±110 psec for no interaction control).

Inhibition of γ-secretase Function Does Not Prevent Association of APP with PS1/γ-secretase.

We investigated whether APP and PS1 interactions would still be observed if γ-secretase function was inhibited. We used the highly potent γ-secretase inhibitor DAPT (Dovey, H. F. et al. J Neurochem 76: 173-181, 2001; Micchelli, C. A. et al., FASEB in press, 2002), or a D257A PS1 mutation which diminishes Aβ and Notch1 processing substantially and appears to be a “dominant negative” in terms of γ-secretase function. The shortened lifetime of FITC (PS1) in the presence of Cy3 labeled APP showed that the PS1 loop epitope is in close proximity to the C-terminus of APP, after each of these manipulations (Table 1). In parallel experiments (sister cultures) we measured Aβ production after DAPT treatment and found a significant decrease in Aβ secretion.

Thus, FLIM analysis revealed that neither Asp mutations nor DAPT treatment prevents interactions between APP and PS1: the fluorescence lifetime was about the same as that seen with untreated wild type PS1 (Table 1). Table one illustrates that Aspartate mutations in PS1 or γ-secretase inhibitors did not prevent APP/PS1 FRET. The table shows summary data of the FLIM assay for PS1 loop-APP C-terminus proximity under baseline conditions and in the presence of manipulations to preclude APP γ-secretase cleavage. If there was no interaction, lifetimes on the order of ˜2600 psec were observed. If FRET was detected, a population with a statistically shorter lifetime (<˜2400 psec) was observed.

TABLE 1

| | FRET Donor | FRET Acceptor | FITC Lifetime (psec) |
|---|---|---|---|
| Condition | FITC labeled | CY3 labeled | mean ± SD |
| WT or | PS1 loop | None | 2600 ± 110 (n = 12) |
| D257A | | | |
| PS1 (control) | | | |
| WT PS1 | PS1 loop | APP 770 | 2290 ± 216** (n = 27) |
| WT PS1 | PS1 loop | APP 770 + DAPT | 2416 ± 118* (n = 9) |
| WT PS1 | PS1 loop | APP770 + WPE31C | 1972 ± 264** (n = 30) |
| D257A PS1 | PS1 loop | APP770 | 2255 ± 264** (n = 26) |

*P < 0.05,
**P < 0.01, compared to non-FRETing control

FRET Analysis of the Proximity Between APP and PS1

To verify that the shortened donor fluorescence lifetimes observed in the FLIM experiments are due to FRET between PS1 and APP labels, we confirmed these observations using an alternative strategy to measure FRET: a photobleach-dequenching assay.

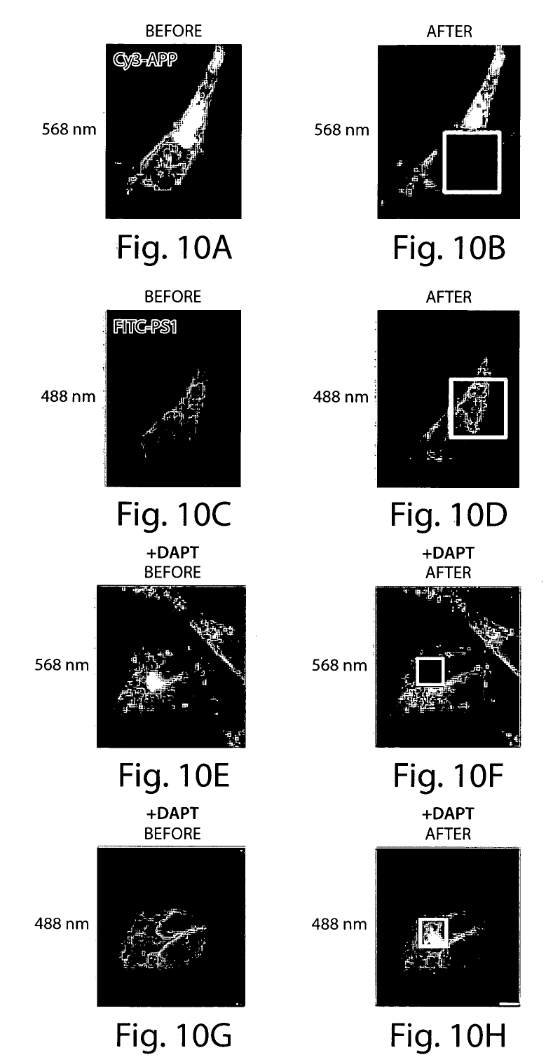

FRET arises when donor and acceptor molecules are <10 nm apart. Cells stably expressing wild type PS1 (wt PS1) and APP770 were double labeled with PS1 loop and APP C-terminal antibodies, followed by FITC- and Cy3-labeled secondary antibodies, respectively. A small area within the cell was photobleached at 568 nm (acceptor only), and FITC fluorescence intensity (at 488 nm) was measured before and after photobleaching. FIGS. 10A-D shows the presence of FRET between PS1 and APP CTF. We observed an 11.6±3% (n=8, P<0.05) increase in FITC fluorescence intensity in the bleached area, indicating that the C-terminus of APP is in close proximity to the loop region of PS1.

Treatment with γ-secretase inhibitors (or Asp mutations in PS1) prevents processing of substrates at γ-secretase site. Therefore, we tested whether FRET would be altered by DAPT treatment. Treatment with DAPT also revealed significant FRET between APP and PS1 (19.8±2%, n=8, P<0.05), indicating that the γ-secretase inhibitor does not disrupt association of APP with the PS1 containing γ-secretase complex (FIGS. 10E-H). This was also true for the D257A PS1 mutation; we observed 25.9±11% (n=8, P<0.05) increase in FITC-fluorescence intensity, compared to a non-FRET control. Therefore, both the FLIM and photobleaching assays suggest that APP interacts closely with PS1 even when γ-secretase is inhibited.

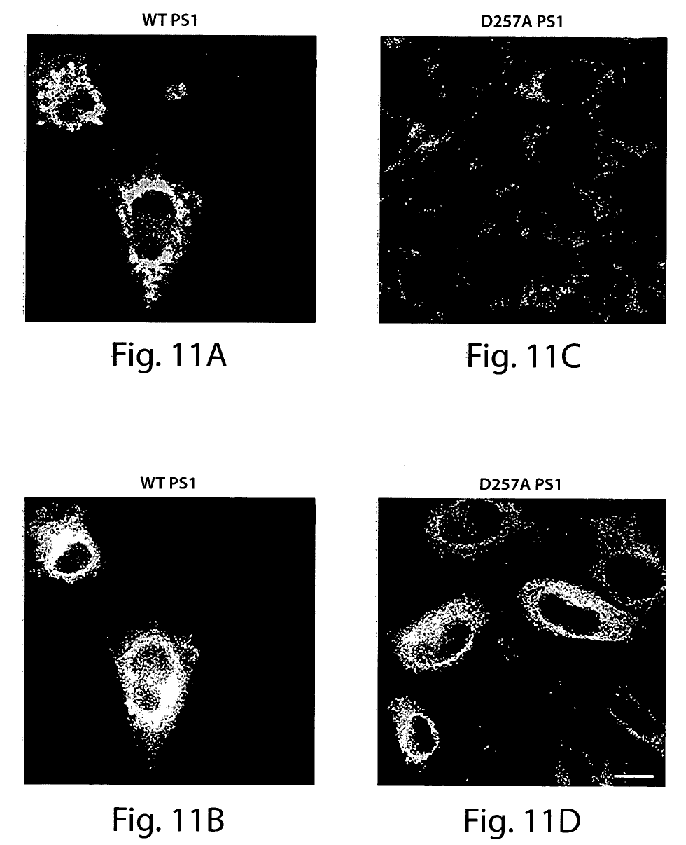

These data suggest either that DAPT and the D257A mutation stabilize an APP-PS1 active site complex (acting as noncompetitive inhibitors), or that there is a second binding site on the PS1/γ-secretase complex for APP. We reasoned that an agent that binds to the active site would compete with APP, blocking the APP-PS1 interaction if the first possibility were the case. We used a well-characterized transition state analogue, WPE-III-31C, that binds to the active site of γ-secretase (Esler, W. P. et al., Proc Natl Acad Sci USA 99: 2720-2725, 2002). The cells were treated with WPE-31C to examine whether APP would still be associated with the PS1 loop region. The data show clearly that, although the active site is occupied, there remains a tight association between APP and PS1 (Table 1). Thus, our data suggest that there is a docking site, distinct from the catalytic site, on the PS1/γ-secretase complex. The association of WPE31C with the active site suggests its possible use as a histological reagent to identify the subcellular location of the active site, and as a FRET reagent to probe the microenvironment near the active site. It has been observed that transition state analogues like WPE31C bind specifically to the heterodimeric form of PS1, and not to the holoprotein prior to cleavage. We therefore expect them not to bind to the D257A mutant protein that does not undergo cleavage. We used a biotinylated form of WPE31C (Esler, W. P. et al., Proc Natl Acad Sci USA 99: 2720-2725, 2002) as a histological reagent. Analysis of wild type PS1 (PS70 cells) or the D257A mutant PS1 CHO cells exposed to biotinylated WPE31C showed a striking difference in CY3-strepavidin distribution. FIG. 11 shows little staining of the D257A cells, which did not co-localize with PS1 immunostaining by a monoclonal antibody developed with FITC. In contrast, wt PS1 expressing cells were strongly stained by WPE31C, and this staining co-localized with PS1 immunostaining. Of note, there was less WPE-31C/Cy3 signal in the proximal perinuclear compartment, compared with PS1 immunoreactivity, confirming our observation that some of this PS1 is in a different conformation and is likely not proteolytically active.

FLIM Analysis of the Biotinylated Transition State Analogue WPE31C Suggests a Close Association with the PS1 Loop Region.

We reasoned that if WPE31C bound to the active site, and if the transmembrane domains near the PS1 loop are near the active site, we might be able to detect an interaction between the ligand (WPE31C-bi) and the PS1-loop region using FLIM-FRET. We examined this possibility in wt PS1 expressing cells, and found that there was strong FRET (as observed by a shortening in fluorescence lifetimes) between the PS1 loop antibody and WPE31C. Interestingly, neither a PS1 N-terminal antibody, nor an APP CT antibody, showed statistically significant FRET with WPE31C (despite complete co-localization of the PS1 N-terminal antibody with WPE31C at the light level), Table 2. Table 2 illustrates that PS1 loop showed the strongest FRET with WPE-31C-biotin. The degree of the fluorescence lifetime shortening indicated that the transition state analogue (γ-secretase active site inhibitor) WPE-31C was in the closest proximity to the PS1 loop region, and far away from PS1 N-terminus. These data reinforce the specificity and proximity dependence of the FLIM assay, and act as an additional negative control for the technique.

TABLE 2

| | FRET Donor | FRET Acceptor | FITC lifetime (psec) |
|---|---|---|---|
| Condition | FITC labeled | CY3 labeled | mean ± SD |
| WT PS1 | PS1 NT (X81) | none | 2630 ± 82 (n = 9) |
| WT PS1 | PS1 loop | WPE 31C-biotin | 1815 ± 162* (n = 15) |
| WT PS1 | PS1 NT (X81) | WPE 31C-biotin | 2672 ± 44 (n = 14) |
| WT PS1 | PS1 CT (R&D) | WPE 31C-biotin | 2220 ± 110* (n = 12) |

*P < 0.001, compared to non-FRETing control

Nicastrin is Important for APP-PS1 Association.

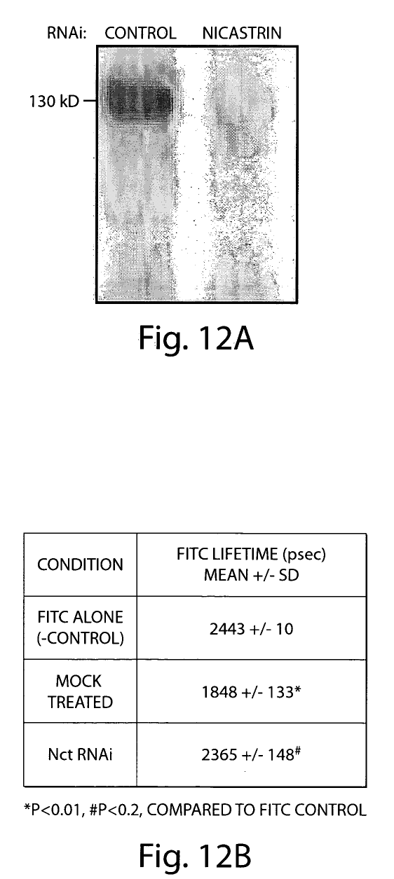

The results of the above-described experiments showed that there is an APP-PS1 interaction even in the presence of pharmacological inhibitors and dominant negative PS1 mutations. Nicastrin has been reported to be a major component of the γ-secretase complex, and nicastrin RNAi blocks γ-secretase function (Edbauer, D. et al., Proc Natl Acad Sci USA 99: 8666-8671, 2002). We tested whether Nicastrin interactions with PS1 and γ-secretase were important for the association of substrates to the γ-secretase complex. We used a Nicastrin RNAi knockdown approach, followed by FLIM analysis, to examine APP-PS1 interaction in HEK 293 cells. Three days after treatment with Nicastrin siRNA, the amount of immunodetectable Nicastrin was minimal as seen by Western blot (FIG. 12A). There was no evidence of enhanced cell death in the treated (compared to mock treated) cells. Nicastrin RNAi treated cells were then immunostained with APP and PS1 antibodies as described above. The proximity between endogenous APP CTF and PS1 loop was then analyzed by FLIM. Importantly, the same pattern of PS1-APP FLIM occurred using endogenous proteins as we had observed in stably transfected CHO cells. In contrast to our earlier studies of D257A PS1 mutations, DAPT, and WPE31C, which did not affect the association of APP with PS1, we found that Nicastrin RNAi treatment completely disrupts the APP/PS1 assembly (FIG. 12B).

Discussion

FRET (or FRET based FLIM) provides information about the proximity between donor- and acceptor-labeled molecules. FRET arises when donor and acceptor molecules are brought into close (<10 nm) proximity upon the formation of a protein complex. Using this “proximity” approach, we studied how different pharmacological agents or genetic manipulations affect APP-PS1 interactions in intact cells. We found that 1) APP-PS1 interactions are tighter near the cell membrane, rather than in the Golgi/ER (despite greater expression and apparent co-localization in the latter compartments); 2) Neither Aspartate mutations nor γ-secretase inhibitors prevent the association of APP with PS1, suggesting the presence of both a docking site and an active site on the PS1/γ-secretase complex; 3) Nicastrin is important for docking of APP to the PS1/γ-secretase. These results confirm and extend the cell free assay findings that APP CTFs and PS1 “pull down” in co-immunoprecipitation experiments (Xia, W. et al., Proc Natl Acad Sci USA 97: 9299-9304, 2000), PS1 and APP CTF co-isolate when bound to a column containing a transition-state analogue (Esler, W. P. et al., Proc Natl Acad Sci USA 99: 2720-2725, 2002), and that isolated γ-secretase shows nonlinear kinetics with various inhibitors (Tian, G. et al., J Biol Chem 277: 31499-31505, 2002). However, in these reconstitution experiments, varying detergent conditions could have altered the extent to which various constituents were present. We show that in intact cells, inhibition of γ-secretase activity either by the introduction of Aspartate mutations in PS1 or by treatment with γ-secretase inhibitors, did not affect association of APP with the PS1/γ-secretase complex. In both cases APP and PS1 still interact at least as strongly as with untreated wild type PS1 (Table 1). Moreover, occupation of the γ-secretase active site by the inhibitor WPE31C, a well-characterized transition state analogue that can be used to isolate a functional γ-secretase complex from cell homogenates (Esler, W. P. et al., Proc Natl Acad Sci USA 99: 2720-2725, 2002), did not prevent association of APP with PS1 in intact cells. These data show clearly that a very tight association between APP and PS1 remains despite active site occupation, which is consistent with the presence of a docking site. In addition, we show that WPE31C, which binds to the active site of γ-secretase, is closely associated with the PS1 loop domain, but not with the N-terminal epitope. These results are consistent with co-purification data (Esler, W. P. et al., Proc Natl Acad Sci USA 99: 2720-2725, 2002), and strongly support the notion that the γ-secretase active site is located between TM6 and TM7 where the two critical aspartates lie, near the loop region of PS1.

Our data also address the subcellular localization of APP-PS1 interactions. The “spatial paradox” reflects the observation that APP and PS1 overlap most strongly in the Golgi and ER, but that γ-secretase function and APP cleavage are not prominent in the site of the greatest overlap—the ER (Cupers, P. et al., J Cell Biol 154: 731-740, 2001). This discrepancy has been used to support the argument that PS1 is not γ-secretase. Our imaging technique confirms that, by conventional immunostaining, APP and PS1 overlap to the greatest extent in a perinuclear location (see FIG. 2). However, they come into closest proximity, as detected by the FLIM assay, in the distal compartments, near the cell surface. We hypothesize that PS1 adopts an active conformation only in distal compartments. If so, these data provide an alternative explanation for the “spatial paradox” in which PS1 in distal cellular compartments is an important component of a γ-secretase complex. We also tested the role of Nicastrin in the formation of an APP/PS1 complex. Nicastrin knockdown using RNAi has been reported to disrupt PS1 processing to heterodimeric forms, and γ-secretase function (Edbauer, D. et al., Proc Natl Acad Sci USA 99: 8666-8671, 2002). We asked if RNAi mediated knockdown of Nicastrin would alter APP-PS1 interactions, since other manipulations that inhibit γ-secretase, including the D257A PS1 mutation that also precludes heterodimer formation, still allow for APP-PS1 interactions that are detectable by co-IP or FRET. Interestingly, we could not detect any FRET between APP and PS1 in cells treated with Nicastrin RNAi. We interpret this result to mean that the absence of Nicastrin diminishes APP-PS1 interactions. Our current data do not differentiate between the possibilities that Nicastrin contributes directly to the docking site, or that it acts upstream of docking in the formation of the γ-secretase complex.

Our model suggests that APP interacts with γ-secretase at a docking site as well as at a proteolytic cleavage site. The docking site may confer specificity or play a modulatory role in γ-secretase-substrate interactions. If APP and Notch (or other substrates) are “docked” at different sites, or if docking of each substrate is subserved by separate docking proteins, it may be possible to selectively inhibit APP-γ-secretase interactions. There have been a number of proteins reported to undergo PS1-dependent γ-secretase cleavage (Baki, L. et al., Proc Natl Acad Sci USA 98: 2381-2386, 2001; Ni, C. Y. et al., Science 294: 2179-2181, 2001; Lee, H. J. et al., J Biol Chem 277: 6318-6323, 2002; Marambaud, P. et al., Embo J 21: 1948-1956, 2002; May, P. et al., J Biol Chem 277: 18736-18743, 2002). Therefore, because direct inhibition of γ-secretase cleavage could disrupt many substrates, a better understanding of specific APP-γ-secretase interactions at a non-catalytic site may ultimately have therapeutic implications, because drugs targeted to a docking site might better discriminate among substrates than active site γ-secretase inhibitors.

### Example 3

Background

Deposition of Aβ containing plaques in the brain is one of the major neuropathological hallmarks of Alzheimer's disease (AD). The final enzymatic step in generating Aβ via intramembranous cleavage of the APP is performed by the PS1 dependent α-secretase complex (Fortini, M. E., Nat Rev Mol Cell Biol 3: 673-684, 2002; Fraser, P. E. et al., Biochem Biophys Acta 1502: 1-15, 2000; Selkoe, D. J., Proc Natl Acad Sci USA 98: 11039-11041, 2001). PS1 is a 467 amino acid eight transmembrane domain protein with its N- and C termini, and a major loop domain (between predicted TM6 and TM7) oriented towards the cytoplasm (Doan, A. et al., Neuron 17: 1023-1030, 1996; Li, X. et al., Neuron 17: 1015-1021., 1996; Li, X. et al., Proc Natl Acad Sci USA 95: 7109-7114, 1998). During maturation PS1 associates with a high-molecular-weight complex and undergoes proteolysis within exon 9, yielding a stable functionally active heterodimeric complex of N- and C-terminal fragments (Capell, A. et al., J Biol Chem 273: 3205-3211., 1998; Levitan, D. et al., Proc Natl Acad Sci USA 98: 12186-12190, 2001; Podlisny, M. B. et al., Neurobiol Dis 3: 325-337, 1997; Seeger, M. et al., Proc Natl Acad Sci USA 94: 5090-5094., 1997; Thinakaran, G. et al., Neuron 17: 181-190, 1996; Yu, G. et al., J Biol Chem 273: 16470-16475,1998). Immature PS1 holoprotein (or PS1 that does not undergo proteolysis due to mutated aspartates in TM6 or TM7 (Wolfe, M. S. et al., Nature 398: 513-517, 1999)) does not support α-secretase activity. Thus, functionally active PS1 must undergo post-translational modification and associate within membrane with at least three other transmembrane proteins (Yu, G. et al., Nature 407: 48-54, 2000; Goutte, C. et al., Proc Natl Acad Sci USA 99: 775-779, 2002; Francis, R. et al., Dev Cell 3: 85-97, 2002). Efforts at structural analysis of the members of the α-secretase complex had not yet been successful.

We investigated whether familial Alzheimer's disease (FAD) PS1 mutations may cause similar molecular alterations in PS1. To examine the sub-domain structure of PS1 in situ in intact cells we optimized a novel fluorescence resonance energy transfer (FRET) microscopy approach, fluorescence lifetime imaging microscopy (FLIM). A FRET signal is observed only if two fluorophores are less than ˜5-10 nm from one another; even if the fluorophores decorate two epitopes of a single protein, the presence and amount of FRET can vary substantially depending on the exact spatial relationship of the donor and acceptor fluorophore. Fluorescence lifetimes of the donor fluorophore were measured as a sensitive way to detect and quantify the extent of FRET between donor and acceptor fluorophores. Lifetime measurements were advantageous for several reasons: (1) The measured lifetimes were fit to two exponential decay curves, representing non-FRETing and FRETing populations, respectively. The bi-exponential fit distinguished the two populations even when the latter represented only a small percent of the total population. Unlike intensity measures, fluorescence lifetimes were not dependent on the concentration of the fluorophore. (2) The extent of shortening of the measured lifetimes provided a quantitative reflection of the FRET efficiency (proximity between the two fluorophores). (3) Any non-FRETing donor (due to mismatch between donor and acceptor fluorophores, lack of specificity of reagents, etc) did not contribute to the measured lifetimes of the FRETing population. Thus, fluorescence lifetime analysis was ideal for detecting spatial signatures of subpopulations of target molecules within the context of living cells.

Methods

Cells and transfections. Primary neurons were prepared as previously described (Berezovska, O. et al., Brain Res Mol Brain Res 69: 273-280, 1999). Briefly, transgenic mice heterozygous for human wild type or M146L mutant PS1 were used (at E16) to prepare primary neuronal cultures from cortex and hippocampus (Berezovska, O. et al., Brain Res Mol Brain Res 69: 273-280, 1999). Neurons were maintained in chemically defined Neurobasal medium (Gibco BRL, MD, USA) containing 2% B27 supplement (Gibco BRL, MD, USA). After two to four days in vitro the cells were immunostained for the FLIM analysis.

Chinese hamster ovary (CHO) cells stably expressing wild-type APP and N2a mouse neuroblastoma cells were used for transient transfections of wild type and FAD mutant PS1;alternatively, CHO cells stably expressing wild type APP and either wild-type PS1 or FAD mutant PS1 were examined (gift from Dr. D. Selkoe, BWH, Boston, Mass.). Superfect reagent (Qiagen Inc., Valencia, Calif.) was used for transient transfections according to the manufacturer instruction.

Antibodies and immunocytochemistry procedures. For the analysis of PS1 conformation, CHO cells transiently or stably expressing wild type or FAD mutant PS1 were double-immunostained with the antibodies against PS1 N-terminus [goat-αPS1 directed against amino acids 14-33 (Sigma-Aldrich, St Louis, Mo.), or x81 directed against amino acids 1-81 (kindly provided by Dr. D. Selkoe, BWH, Boston, Mass.)], and against PS1 C-terminus [rabbit S182 antibody, amino acids 450-467 (Sigma), or C-20 antibody (Santa Cruz Biotechnology, Inc.)]. For the analysis of PS1-APP interactions the cells were immunostained with antibody against APP C-terminus (C8 antibody raised against amino acids 676-695 of APP (Selkoe, D. et al., Proc Natl Acad Sci USA. 85: 7341-7345, 1988)) and antibody to major loop between PS1 TM6 and TM7 [mouse-αPs1, amino acids 263-378 (Chemicon, Temecula, Calif.)]. Pairs of primary antibodies were labeled with secondary antibodies conjugated to FITC or Cy3 for the FLIM analysis.

Fluorescence Lifetime Imaging Microscopy, FLIM, Assay.

Double immunostained cells were used for the FLIM analysis as described previously (Berezovska, O. et al., J Neurosci 23: 4560-4566., 2003). Briefly, fluorescence lifetime of a donor fluorophore (FITC) was measured in the absence of FRET (Cy3 acceptor fluorophore is absent or is more than 10 nm away for the FITC donor, negative control). If the two fluorophores are less than 5-10 nm apart, FRET occurs and the donor fluorophore lifetime shortens. In each experiment, negative controls consisted of cells immunostained with donor fluorophore only or double immunostained with antibodies against antigens known not to interact (no FRET present), such as PS1 and GRP78 BiP (StressGen Biotechnologies, Victoria, BC Canada). Positive controls included Cy3-anti-FITC immunostained cells (Berezovska, O. et al., J Neurosci 23: 4560-4566., 2003). The FLIM software uses multiexponential fluorescence decay curves to calculate FITC fluorescence lifetimes in each pixel of the image. The fluorescence lifetimes are fitted to two curves, representing “non-FRETing” population with a longer lifetime (t1) and “FRETing” population with shorter lifetime (t2). The t2 values are presented in the tables (for the negative control, non-FRETing population, t1=t2). A femto-second pulsed Ti:Sapphire laser (Mai-Tai, Spectra Physics) at 800 nm coupled to BioRad Radiance 2000 microscope was used for multiphoton fluorescence excitation. FITC fluorescence was acquired using emission filter centered at 515/30 nm. Fluorescence lifetimes were recorded using a high speed photomultiplier tube with FWHM response time about 50 psec ((MCP R3809, Hamamatsu) and a fast time-correlated single photon counting acquisition board (SPC-830, Becker&Hickl, Berlin, Germany), that allowed high temporal resolution lifetime acquisition with high spatial resolution imaging (Bacskai, B. J. et al., Journal of Biomedical Optics 8: 368-375, 2003). A 128×128 pixel matrix was created with the single (for FITC alone) or multi-exponential (for FITC and Cy3 double immunostained cells) curve fit data for each pixel, allowing color coding by lifetime.

Results and Discussion

Establishment of a PS1 Subdomain Conformation Assay.

The goal was to develop an assay in which the inactive holoprotein did not show a signal in the FRET analysis, while the catalytically active heterodimer achieved a conformation in which FRET occurred. Multiple different pairs of epitopes on PS1, and multiple donor-acceptor fluorophore pairs were empirically examined. Initially, CHO cells transiently transfected with wild type PS1 (which would be expected to contain both holoprotein and heterodimer) and cells transfected with the D257A PS1 mutant which cannot form heterodimeric, active conformations were used. In cells immunostained with antibodies against PS1 N-terminus (NT) and C-terminus (CT), providing donor and acceptor fluorophores respectively, two major populations of donor lifetimes were detected with the first having a lifetime of ˜2300 psec (equivalent to a non-FRETing donor fluorophore) and the second a statistically shorter lifetime (˜1700 psec), suggesting the presence of FRET and close proximity between the N- and C-termini of the PS1 molecule. Importantly, PS1 molecules which were prevented from generating heterodimeric forms (e.g. D257A mutants or cells treated with Nicastrin RNAi (Wolfe, M. S. et al., Nature 398: 513-517, 1999; Edbauer, D. et al., Proc Natl Acad Sci USA 99: 8666-8671, 2002) did not show a second population with shortened lifetimes, suggesting that the FRET signal (i.e., the molecules giving rise to lifetimes less than the baseline) derived only from PS1 molecules in the heterodimeric conformation. Negative controls, included the examination of lifetimes of PS1 FITC stained without an acceptor present, or PS1 FITC stained with an acceptor present on GRP78-BiP, which co-localizes with PS1 in the endoplasmic reticulum, but is too distant to support FRET. In both instances only non-FRETing FITC lifetimes of ˜2300-2400 psec were observed.

FAD PS1 Mutations Change PS1 Conformation

Numerous FAD mutations in PS1 have been identified since the discovery of the PS1 gene (Sherrington, R. et al., Nature 375: 754-760, 1995 ) (www.alzforum.org). Although it is known that virtually all PS1 mutations cause a change in APP processing with increased production of the longer, highly fibrillogenic form of Aβ, it remains unclear how missense mutations scattered throughout the entire molecule and even a large deletion (Δ exon 9) mutation, that precludes cleavage of the holoprotein, all lead to the same phenotype. To test whether AD-linked PS1 mutations located throughout the entire PS1 sequence lead to a consistent change in PS1 conformation we transiently transfected CHO cells with wild type PS1 or each of six familial AD PS1 missense mutations located in different domains of the PS1 molecule: M139V and M146L mutations in transmembrane domain (TM) (Fraser, P. E. et al., Biochem Biophys Acta 1502: 1-15, 2000); L166P mutation in TM3, L286V mutation, in the hydrophilic loop, between TM6 and TM7, G384A mutation in TM7 and C410Y mutation in TM8. All mutations increase Aβ42/Aβ40 ratio and lead to early onset autosomal dominantly inherited AD (Scheuner, D. et al., Nature Med 2: 864-870,1996; Borchelt, D. et al., Neuron 17: 1005-1013, 1996). Immunostaining of the cells expressing either wild type PS1 or FAD mutant PS1 did not show any difference in the localization of PS1 immunoreactivity. FLIM lifetime decay measurements were fit to two exponential decays, the first (longer) equivalent to the non-FRETing population and the second (shorter) reflecting the FRETing population. The fluorescence lifetimes of the second population representing the spatial conformation of only heterodimeric, catalytically active species were used for all subsequent analyses. A dramatic difference in the FRET measures was observed between wild type and each of the FAD PS1 mutants (Table 3) with shorter lifetimes observed for each mutant PS1. This suggests that the orientation of the N- and C-termini are altered with respect to one another in all tested FAD missense mutants compared to wild type PS1.

TABLE 3

| | FITC lifetime |
|---|---|
| Condition | (t2, mean ± SD, psec) |
| PS1 wild type (n = 55) | 1768 ± 141 |
| PS1 M139V (n = 9) | 1144 ± 54* |
| PS1 M146L (n = 36) | 1191 ± 342* |
| PS1 L166P (n = 23) | 830 ± 191* |
| PS1 L286V (n = 63) | 1054 ± 285* |
| PS1 G384A (n = 30) | 849 ± 124* |
| PS1 C410Y (n = 37) | 1162 ± 404* |
| PS1 Δ ex9 (n = 36) | 1019 ± 221* |

(n = cell number; *P < 0.01 compared to wild type PS1).

Table 3 illustrates differences in the proximity between N- and C-termini in wild type PS1 and FAD mutant PS1. A time-domain FLIM and a bi-exponential decay curve fitting were used to calculate average FITC lifetimes (t1 and t2) in each cell. First, FITC lifetime (t1) in the absence of FRET (in PS1 NT-BiP double immunostained cells, negative control) was calculated using FLIM software. FITC-IgG lifetime (t1) was 2321±59 psec. To analyze PS1 NT and CT proximity, average FITC lifetimes per cell were fitted to two curves representing non-FRETing population (fixed as t1) and remaining (t2) average FITC lifetime per cell was recorded and presented in the table. A (t2) lifetime shorter than (t1) lifetime implies the presence of FRET, and a close proximity between the donor and acceptor. For the analysis of the effect of FAD PS1 mutations CHO cells were transiently transfected with either wild-type PS1 or FAD mutant PS1. One-way ANOVA was performed to analyze differences in the lifetime followed by least significant difference post hoc analysis. Levene's test was also performed to determine whether variances were equal.

PS1 is known to require proteolytic cleavage within exon 9 to have catalytic activity. Paradoxically, a familial AD linked mutation causes the deletion of exon 9 but results in a catalytically active protein that does not undergo endoproteolysis (Borchelt, D. et al., Neuron 17: 1005-1013, 1996; Perez-Tur, J. et al., Neuroreport 7: 297-301, 1995). We predicted that the conformation of PS1 Δex9 would be similar to the wild type active heterodimer, rather than the inactive PS1 holoprotein. Indeed, analysis of PS1 Δex9 expressing cells revealed FRET between the N- and C-termini of PS1, consistent with the conformation of the wild type heterodimer (and unlike the wild type holoprotein). Moreover, the degree of FITC lifetime shortening in PS1 .ex9 exceeded that of wild type PS1, and was similar to the other FAD missense mutations tested (Table 3).

Next we verified that the effect of FAD mutations on PS1 conformation is not a result of transient transfection and is not a cell-specific phenomenon. Indeed, the effect of FAD PS1 mutations on PS1 conformation was similar in CHO and in N2a neuroblastoma cells transiently transfected with wild type PS1 and FAD mutant PS1 plasmids, as well as in stably transfected CHO cell lines when wild type PS1 expressing cells were compared to FAD PS1 expressing cells. We also confirmed these observations in primary neurons derived from transgenic mice expressing either human wild type PS1 or M146L PS1 (Berezovska, O. et al., Brain Res Mol Brain Res 69: 273-280, 1999). In addition, all observations were confirmed using two different sets of antibodies to PS1 NT and to PS1 CT.

Effect of PS1 FAD Mutations on APP-PS1 Interactions.

Because FAD mutations in PS1 lead to a shift in the site preference of APP α-secretase cleavage from the Aβ40 to the Aβ42 position, we postulated that alterations in PS1 conformation might lead to a difference in APP-PS1 interactions. We used FLIM assay of APP-PS1 interactions we recently developed based on the proximity of APP CT and PS1 loop domains (Berezovska, O. et al., J.Neurosci 23: 4560-4566., 2003) to determine whether the PS1 mutations affected this interaction. We found that the six FAD PS1 mutations tested (Δex9 mutation lacks the loop epitope) displayed a decreased measured fluorescence lifetime, reflecting an increase in the proximity of the PS1-loop to the APP CT, as compared to wild type PS1 (Table 4). Table 4 shows the effect of PS1 FAD mutations on the proximity between APP C-terminus and PS1 loop domain. CHO cells were transiently transfected with either wild type PS1 or FAD mutant PS1. FITC-IgG lifetime in the absence of FRET was 2347±35 psec, n=96.

TABLE 4

| | FITC Lifetime P value |
|---|---|
| | (compared to (t2, mean ± |
| Condition | SD, psec) wild type PS1) |
| PS1 wild type (n = 120) | 1872 ± 306 — |
| PS1 M139V (n = 12) | 1520 ± 206 P < 0.01 |
| PS1 M146L (n = 40) | 1725 ± 370 P = 0.05 |
| PS1 L166P (n = 14) | 1586 ± 158 P < 0.04 |
| PS1 L246V (n = 40) | 1645 ± 393 P < 0.001 |
| PS1 G384A (n = 11) | 1641 ± 329 P < 0.05 |
| PS1 C410Y (n = 45) | 1427 ± 365 P < 0.001 |

(n = cell number; ANOVA).

By labeling different domains of a complex multipass transmembrane protein, PS1, we were able to study the relative proximity of those domains to one another in situ in intact cells, using a highly sensitive FLIM assay. We found two populations of donor fluorophore lifetimes in cells immunostained with antibodies against PS1 NT and CT: non-FRETing with PS1 NT far apart from PS1 CT, representing immature PS1 holoprotein, and FRETing population with the NT-CT distance less than 10 nm, representing functionally active PS1 heterodimer. These data suggest that the two populations are in different conformational states.

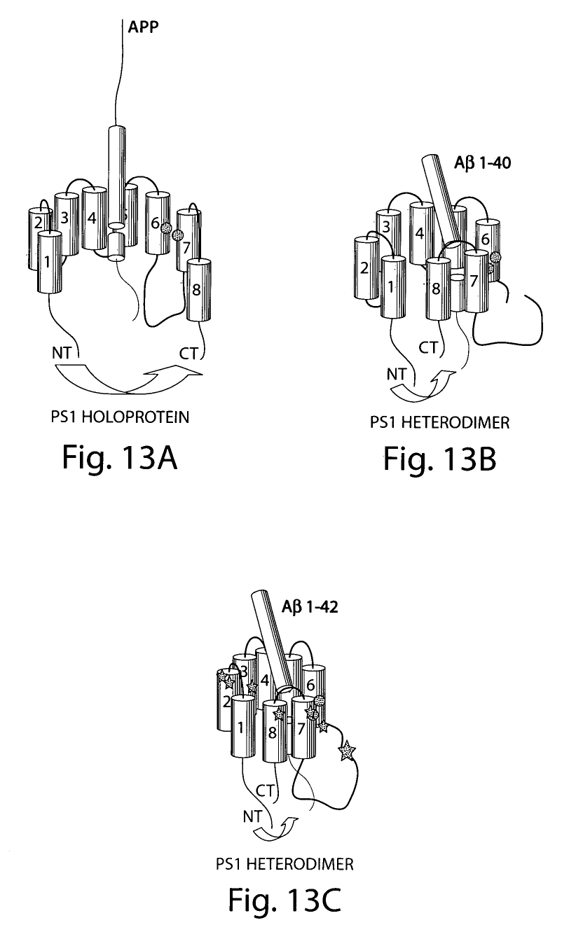

We found that all FAD mutations tested (both missense and deletion) located in the N-terminal domain, mid-regions, or C-terminal domain of the PS1 molecule lead to a common shift in PS1 conformation: closer interactions between PS1 NT and CT (FIG. 13). Furthermore, we show that this change in PS1 conformation then leads to an alteration of the structure of the PS1-APP complex and affects how APP substrate is presented to the active site of α-secretase. We propose that this change in the alignment of APP substrate with PS2/α-secretase accounts for the shift in Aβ cleavage site between position 40 and 42 (FIG. 13).

FIG. 13 provides a schematic representation of the predicted PS1 conformation for immature PS1 holoprotein (FIG. 13A), and functionally active PS1 heterodimer (FIG. 13B and C), which exists in a dynamic state. FAD mutations in PS1 (or some other unknown factors) shift the equilibrium to favor (FIG. 13C) conformation and change the alignment of the APP with the active site of PS1/gamma-secretase to favor cleavage at Aβ42 position on APP. FITC labeled PS1 NT and Cy3 labeled PS1 CT are shown; ● in TM domains 6 and 7 indicate the position of Aspartate 257 and Aspartate 385, respectively (a putative catalytic site); ★ in (c) indicate the position of analyzed FAD PS1 mutations (small ★: M139V, M146L, L166P, L286V, G384A, C410Y, and large ★: exon 9 deletion). APP C99 substrate is aligned with PS1 to be predominantly cleaved at Aβ40 (FIG. 13B) or Aβ42 (FIG. 13C) position. For simplicity, only one component of the multimeric α-secretase complex is shown.

### Example 4

Background

Amyloid-β (Aβ) peptide is a 40-42 residue peptide believed to play a central role in the pathogenesis of Alzheimer disease (AD) (Sisodia, S. S. & St George-Hyslop, P. H., Nat. Rev. Neurosci. 3, 281-290, 2002). Aβ is generated from a larger protein called the β-amyloid precursor protein (APP) that undergoes a series of endoproteolytic events, ultimately liberating the small fragment. The last cleavage involves an unusual form of proteolysis through which APP is cleaved by γ-secretase within the transmembrane domain at residue +40 or +42, releasing Aβ₄₀ or Aβ₄₂ respectively. Around 90% of the secreted Aβ peptides are Aβ₄₀, which is soluble and fairly innocuous, and the remaining species are Aβ₄₂₋₃, which are highly fibrillogenic and are deposited selectively in amyloid plaques in AD. (Hardy, J. & Selkoe, D. J., Science 297, 353-356, 2002). Presenilin (PS) and at least three other components, nicastrin, aph-1 and pen-2, have been identified as required members of the ˜250 kDa γ-secretase complex (Yu, G. et al., Nature 407: 48-54, 2000; Kimberly, W. T. et al., Proc Natl Acad Sci USA 100: 6382-6387, 2003; Goutte, C. et al., Proc Natl Acad Sci USA 99: 775-779, 2002; Francis, R. et al., Dev Cell 3: 85-97, 2002).

Recent reports have shown that, surprisingly, some nonsteroidal anti-inflammatory drugs (NSAIDs) preferentially lower Aβ₄₂ in cell culture and in transgenic mice(Weggen, S. et al., Nature 414: 212-216, 2001; Morihara, T. et al., J Neurochem 83: 1009-1012, 2002; Lim, G. P. et al., J Neurosci 20: 5709-5714, 2000; Jantzen, P. T. et al., J Neurosci 22: 2246-2254, 2002; Yan, Q. et al., J Neurosci 23: 7504-7509, 2003; Eriksen, J. L. et al., J Clin Invest 112: 440-449, 2003). However, the reduction in Aβ is independent of cyclooxygenase (COX) activity, the accepted target of NSAIDs. This effect of NSAIDs appears to be to change APP metabolism to generate more soluble Aβ₃₈ and less Aβ₄₂ (Weggen, S. et al., Nature 414: 212-216, 2001; Weggen, S. et al., J Biol Chem 278: 31831-31837, 2003), but the mechanism remains unknown.

We have used a novel fluorescence lifetime imaging microscopy (FLIM) technique to show that the reduction in Aβ₄₂ observed with NSAIDs is associated with a change in the conformation of PS1 in vitro and in vivo. The change in PS1 conformation was observed only for those NSAIDs with marked effects on Aβ₄₂ production. Further experiments support the conclusion that NSAIDs lead to a change in the way that APP is presented to PS1, perhaps leading to a shift in the cleavage site.

We applied FLIM and monitored presenilin/γ-secretase conformation, and have demonstrated that Aβ42 lowering NSAIDs specifically alter presenilin 1 conformation in a consistent fashion. In addition, the same drugs affect the interaction between APP and presenilin1, also monitored by fluorescence lifetime imaging. These effects on presenilin/γ-secretase conformation were not observed with conventional γ-secretase inhibitors. Our results indicated that specific NSAIDs alter presenilin 1 conformation, leading to a change in APP cleavage products. This observation provides novel insights on the mechanism of action of NSAIDs and a novel approach for developing drugs acting at allosteric sites.

Methods

Cell culture and drug treatment: PS70 cells, CHO cells stably overexpressing wild-type human PS1 and APP 751 (Xia, W. et al., J. Biol. Chem. 272, 7977-7982, 1997), mouse N2a neuroblastoma, or human embryonic kidney (HEK) cells were used in this study. Cells were cultured in OPTI-MEMI with 5% fetal bovine serum at 37° C. with 5% CO₂ in a tissue culture incubator. Primary cultured hippocampal-cortical neurons were prepared from embryonic day 16-18 mouse brain as described (Berezovska, O. et al., Brain Res Mol Brain Res 69: 273-280, 1999).

The NSAIDs (S)-ibuprofen (250 mM, Biomol, Plymouth Meeting, Penn.), (R)-ibuprofen (250 mM, Biomol), and flurbiprofen (250 mM, Calbiochem, San Diego, Calif.) were dissolved in 100% ethanol. Meloxicam (50 mM, Biomol), naproxen (100 mM, Biomol) and indomethacin (50 mM, Biomol) were dissolved in DMSO and aspirin (1 M, Sigma-Aldrich, St. Louis, Mo.) was dissolved in distilled water. DAPT and WPE-III-31C (a gift from M. Wolfe, Brigham and Women's Hospital, Boston, Mass.) were dissolved in DMSO. Cells were plated onto either 12-well plates (for Aβ determinations) or onto 4-chamber slides (FLIM studies) and treated for 24 hours with NSAIDs prior to the analysis. A vehicle control chamber treated with ethanol or DMSO (0.1%) was included in each experiment. Cell toxicity was analyzed for all drugs tested by measuring adenylate kinase levels using Toxi-light reagent (Camdex, New Jersey).

Transfection protocol: Transient transfection in cells was performed using Superfect reagents (Qiagen, Valencia, Calif.) according to manufacturer's instructions. Primary cultured neurons were transfected with wild-type PS1 plasmid using the method of calcium phosphate co-precipitation as described (Berezovska, O. et al., Brain Res Mol Brain Res 69: 273-280, 1999; Chen, C. et al., Biotechniques 6: 632-638, 1987).

ELISA, BACE-specific enzymatic activity assay and Western Blots: For Aβ ELISA we used 500 μl of conditioned medium collected 24 hours post-treatment. Formic acid-extractable Aβ in the mouse brain was measured as described (Fukumoto, H.,et al., Am. J. Pathol. 164, 719-725, 2004). The capture antibody was BNT77 and the detection antibody was horseradish peroxidase (HRP)-conjugated BA27 for Aβ40 and HRP-conjugated BC05 for Aβ₄₂ (Asami-Odaka, A. et al., Biochemistry 34: 10272-10278, 1995). We used BAN50 as a capture antibody and HRP-conjugated BNT77 as a detection antibody for Aβ₁₋ₓ (a gift from Takeda, Japan) (Asami-Odaka, A. et al., Biochemistry 34: 10272-10278, 1995). BACE activity was measured using a sensitive BACE-specific enzymatic activity assay (Fukumoto, H.,et al., Am. J. Pathol. 164, 719-725, 2004).

For the Western Blot analysis, the cells were lysed in 1% Triton X-100 and electrophoresed in 10-20% SDS-polyacrylamide Tris-Glycine gels. The immunoblotting was performed using the APP C8 antibody (Selkoe, D. J. et al., Proc. Natl. Acad. Sci. USA 85, 7341-7345, 1988), mouse actin (Sigma-Aldrich) or rabbit BACE1 (Calbiochem), followed by detection with ECL reagent (Perkin Elmer, Boston, Mass.). Bands on films were quantitated (Odyssey software, LI-COR, Nebraska) and the values normalized to actin expression.

Fe65-dependent APP luciferase transactivation assay: HEK cells were cotransfected with the APP-Gal4 construct, pG5E1B-luc reported plasmid (courtesy of Dr. Südhof, University of Texas) and Fe65 (Cao, X. & Sudhof, T. C., Science 293, 115-120, 2001).

Immunofluorescent staining: Cells were fixed and immunostained after treatment with NSAIDs as described (Jack, C. et al., Brain Res Mol Brain Res 87: 166-174, 2001). For the FLIM assay for PS1-APP proximity (Berezovska, O. et al., J. Neurosci. 23, 4560-4566, 2003), we used APP C-terminal antibody C8 and a PS1 loop antibody (Chemicon, Temecula, Calif.). For the in vitro FLIM assay of proximity between the N- and C-termini (NT and CT respectively) of PS1 we used two sets of antibodies (NT X81, a gift from Dr. Selkoe, and CT, R&D Systems, Minneapolis, Minn. or NT and CT S182, Sigma-Aldrich, St. Louis, Mo.). For the in vivo FLIM assay of proximity between the N- and C-termini of PS1 we used a NT antibody (Sigma-Aldrich) and a CT 4627 antibody (Podlisny, M. B. et al., Neurobiol. Dis. 3, 325-337, 1997. For both FLIM assays Cy3-labeled secondary antibody was used as an acceptor fluorophore and a FITC-labeled secondary antibody was used as a donor fluorophore.

Ibuprofen treatment of wtPS1 mice: Three-month-old transgenic mice over-expressing wtPS1 were treated with S-ibuprofen (375 ppm in diet, Research Diets, New Brunswick, N.J.) or a control diet for 15 days (6 mice per group). There were no differences in the amount of chow consumed or in the mouse weight between treatment groups. At the end of the experimental period animals were sacrificed by CO₂ inhalation. The brains were dissected, half of each brain was frozen on dry ice for Aβ ELISA and BACE activity analysis and the other half was fixed in 4% paraformaldehyde for immunohistochemistry.

FLIM assay: FLIM has been recently described as a useful technique for the analysis of protein-protein proximity (Berezovska, O. et al., J Neurosci 23: 4560-4566, 2003; Bacskai, B. J. et al., J Biomed Opt 8: 368-375, 2003). The technique is based on the observation that fluorescence lifetimes are shorter in the presence in close proximity (<10 nm) of a FRET acceptor. This property is proportional to the distance between the fluorophores at R⁶. A mode-locked Ti-sapphire laser (Spectra-Physics, Fremont, Calif.) put out a femtosecond pulse every 12 nanoseconds to excite the fluorophore. A high-speed Hamamatsu (Bridgewater, N.J.) detector and a fast time-correlated single photon counting acquisition board (SPC-830, Becker and Hickl, Berlin, Germany) were used to measure fluorescence lifetimes. FITC fluorescence was acquired using emission filter centered at 515/30 nm. Donor fluorophore (FITC) lifetimes were fit to two exponential decay curves representing the “non-FRETing” population with a longer lifetime (t2) and the “FRETing” population with shorted lifetime (t1). The t1 values are presented in the tables (for the negative control, non-FRETing population t2=t1) (Berezovska, O. et al., J. Neurosci. 23, 4560-4566, 2003; Bacskai, B. J., et al., J. Biomed. Opt. 8, 368-375, 2003).

As a negative control, the donor fluorophore (FITC) lifetime was measured in the absence of the acceptor (Cy3). Cells were immunostained with primaries antibodies, followed only by a FITC-labeled secondary antibody. As a positive control, FITC lifetime was measured in the presence of a FRET acceptor in close proximity (Berezovska, O. et al., J. Neurosci. 23, 4560-4566, 2003).

Statistical analysis: One-way ANOVA was performed to analyze differences in lifetime followed by least significant difference post hoc analysis. Levene's test was also performed to determine whether variances were equal.

Results

NSAIDs Lower Aβ₄₂ without Decreasing Aβ₁₋ₓ

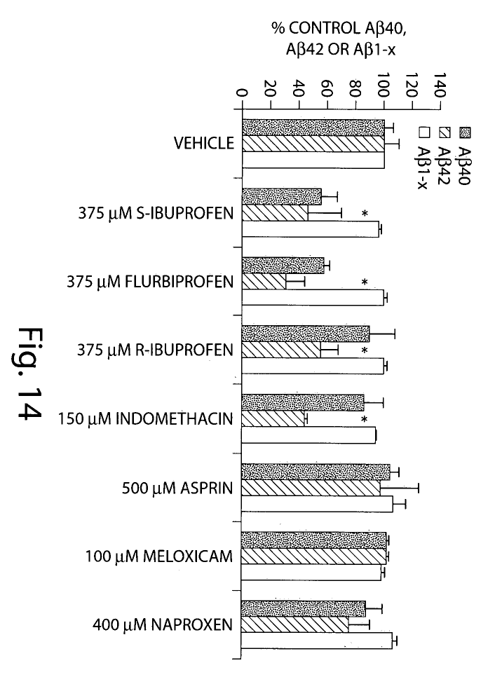

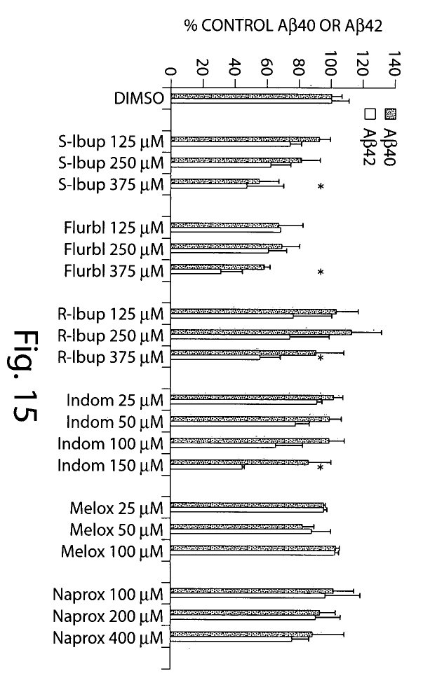

Weggen et al showed that several NSAIDs reduce production of Aβ₄₂, favoring the generation of shorter species (Weggen, S. et al., Nature 414: 212-216, 2001). We examined the mechanism whereby this occurs. We first demonstrated that both COX inhibitors and inactive COX inhibitor enantiomers have an effect on Aβ metabolism. We treated Chinese hamster ovary (CHO) cells stably transfected with APP and PS1 (PS70 cells) with several NSAIDs at different doses for 24 hours and measured Aβ₄₀ and Aβ₄₂ using ELISA (FIG. 14). We observed a marked reduction in Aβ₄₂ in cells treated with S-ibuprofen, R-ibuprofen, indomethacin, and flurbiprofen. S-ibuprofen and flurbiprofen also reduced Aβ₄₀ compared to untreated cells. The effect on Aβ₄₂ was dose-dependent for all active NSAIDs (see FIG. 15). No or small change in Aβ₄₂ or Aβ₄₀ was observed with aspirin, meloxicam or naproxen. No signs of toxicity, assessed by adenylate kinase levels, were observed at the doses tested in CHO or mouse N2a neuroblastoma cells.

In order to determine whether the reduction of Aβ₄₀ and/or Aβ₄₂ reflected a reduction in total Aβ production or a shift to shorter species (Weggen, S. et al., Nature 414: 212-216, 2001), we measured Aβ₁₋ₓ. We predicted that if the changes in Aβ₄₂ and Aβ₄₀ reflected a shift toward other species, Aβ₁₋ₓ should remain unchanged. Indeed, we did not observe a change in Aβ₁₋ₓ after 24 hours of treatment with high doses of S-ibuprofen, flurbiprofen, indomethacin, naproxen or meloxicam (FIG. 14). These data are in general agreement with previous observations (Weggen, S. et al., Nature 414: 212-216, 2001; Morihara, T. et al., J Neurochem 83: 1009-1012, 2002; Eriksen, J. L. et al., J Clin Invest 112: 440-449, 2003), and suggest that an alteration of γ-secretase cleavage site, rather than inhibition, led to the observed effects on Aβ₄₂.

NSAIDs do not Inhibit BACE or γ-secretase and Preserve APP Intracellular Domain (AICD) Generation

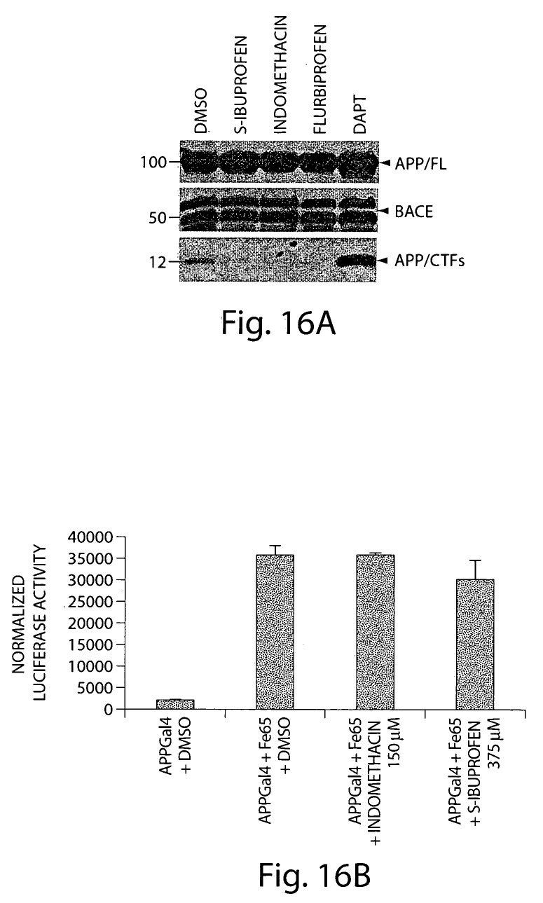

We further examined whether NSAIDs lead to a decrease in γ-secretase activity. Inhibition of γ-secretase activity leads to accumulation of C99 and C83 (APP CTFs) and decreased release of AICD. Mouse N2a neuroblastoma cells were transfected with human APP751, and were treated with different NSAIDs and a Western blot was performed for full-length APP and APP CTFs (FIG. 16A). After quantification we did not see any change in expression of full-length APP and APP CTFs, further suggesting that the reduction in Aβ levels was not due to a reduction in γ-secretase activity. Next we asked whether NSAIDs alter generation of AICD and examined the effects of NSAIDs on the ability of APP-Gal4 to be cleaved by γ-secretase, releasing the Gal4-AICD fragment which activates a Gal4-dependent, artificially expressed, luciferase plasmid (Cao, X. et al., Science 293: 115-120, 2001). It has been shown that the presence of the Fe65 adaptor protein is a requisite for the stabilization of the AICD fragment and its subsequent nuclear translocation for luciferase activation (Cao, X. et al., Science 293: 115-120, 2001). In order to test any effects of NSAIDs on downstream activation by APP-Gal4 we cotransfected HEK cells with APP-Gal4, pG5E1B-luc, Fe65 and β-galactosidase and treated the cells 24 hours with different NSAIDs. We observed an expected increase in the ability of APP-Gal4 to stimulate transcription when Fe65 is cotransfected as compared to an empty vector control (FIG. 16B). After 24 hours of treatment with NSAIDs we did not detect any change in luciferase activity in cells treated with NSAIDs compared to those treated with DMSO. Since it has been suggested (Sastre, M. et al., J Neurosci 23: 9796-9804, 2003), that, under some circumstances, NSAIDs alter β-secretase (BACE) expression, we performed a Western blot analysis of the BACEβ protein expression in cells treated with different NSAIDs. We did not observe any changes in the expression of BACE1 protein in cells treated with NSAIDs compared to that of a vehicle control (FIG. 16A).

We interpret these data to suggest that NSAIDs noncompetitively alter APP-γ-secretase interactions to produce a change in where the cleavage of APP occurs, rather than whether or not cleavage occurs. We reasoned that an alteration in the configuration of γ-secretase-APP interaction might lead to the observed outcome.

NSAIDs Alter APP-PS1 Interactions

To test the possibility that NSAID-mediated change in γ-secretase-APP interactions leads to the change in site of APP cleavage, we used a recently developed fluorescence lifetime-based assay to detect APP-PS1 interactions (Berezovska, O. et al., J Neurosci 23: 4560-4566, 2003). This assay monitors the proximity of the APP C-terminus to the PS1 loop domain, which is adjacent to the putative catalytic site containing transmembrane domain 7 and 8. The FLIM assay is based on the observation that fluorescence lifetimes of a donor fluorophore are shortened in the presence of a FRET acceptor fluorophore in a close proximity (<10 nm). The change in fluorescence lifetime represents a measure of proximity and the results can be displayed in picoseconds or in a pseudocolor-coded image with pixel-by-pixel high spatial resolution.

We first measured fluorescence intensity and FITC fluorescence lifetime in the presence of different NSAIDs to rule out autofluorescence or a direct effect of these drugs on FITC lifetime. Meloxicam was strongly autofluorescent with a lifetime of 324 psec and was excluded from the FLIM experiments. Fluorescence was not observed for the other NSAIDs, and no differences in FITC lifetime were seen in the presence of NSAIDs (FITC+375 μM S-ibuprofen 2372±23 psec; FITC+150 μM indomethacin 2367±9 psec; FITC+400 μM naproxen 2381±38 psec; FITC+1 mM aspirin 2360±26). Neither DMSO nor ethanol (0.1%) changed FITC lifetime.

As a measure of proximity between APP and PS1 we stained the loop region of PS1 and the C-terminus of APP with cyanine 3 (Cy3) and FITC-labeled antibodies respectively, as described (Berezovska, O. et al., J Neurosci 23: 4560-4566, 2003). The FLIM assay showed that these two epitopes are in close proximity mainly at the cell surface, reflected as a shortening in FITC lifetime in cells double immunostained for PS1 and APP compared to FITC lifetime without an acceptor present (Table 5). We did not detect any change in the pattern of expression of APP in CHO cells immunostained with the APP C8 antibody and treated with NSAIDs or γ-secretase inhibitors (data not shown). We have shown previously using FLIM that agents that block APP-PS1 interaction increase FITC lifetime towards the nonFRETing values, ˜2300 psec (Das, C. et al., J Am Chem Soc 125: 11794-11795, 2003). Here we demonstrate that treatment with 375 μM ibuprofen or 375 μM flurbiprofen increased FITC lifetime compared to that of vehicle treatment (Table 5). This effect was not seen with 500 μM aspirin. Our interpretation of these data is that flurbiprofen and ibuprofen, which diminish generation of Aβ₄₂, alter the relative proximity of epitopes on APP and PS1, whereas aspirin, which does not change Aβ₄₂ generation, does not alter PS1-APP proximity. The absence of FRET between the APP C-terminus epitope and the PS1 loop epitope in the presence of specific NSAIDs cannot distinguish between the possibilities that the NSAIDs block APP-PS1 interactions or that they change the conformation of APP or PS1/γ-secretase enough to move the epitopes farther apart. The observation that these NSAIDs do not alter total Aβ generation, AICD generation or Notch processing (Weggen, S. et al., Nature 414: 212-216, 2001), argue strongly against the first possibility.

Table 5 illustrates that NSAIDS alter APP-PS1 interactions. We used a FRET-based assay of proximity between APP and PS1 (Berezovska, O. et al., J. Neurosci. 23, 4560-4566, 2003), in which the C-terminal region of APP (APP-CT) is labeled with a FITC-labeled antibody. In the absence of a FRET acceptor FITC lifetime is ˜2400 psec. In the presence of a FRET acceptor, Cy3-labeled antibody against the loop region of PS1, FITC lifetime is shortened to ˜2100 psec. After treatment with S-ibuprofen or flurbiprofen, but not aspirin, FITC lifetime is significantly longer reflecting an increase in the relative distance of epitopes on APP and PS1. A representative experiment is shown (total number of experiments=5).

TABLE 5

| | FRET | FRET | FITC lifetime | P* value |
|---|---|---|---|---|
| | donor | acceptor | APP-CT | compared |
| Condition | FITC-labeled | Cy3-labeled | (mean ± SD) | to vehicle |
| DMSO (n = 31) | APP-CT | loop PS1 | 2127 ± 224 | — |
| 375 μM S-ibuprofen (n = 18) | APP-CT | loop PS1 | 2307 ± 107 | P = 0.001 |
| 375 μM flurbiprofen (n = 11) | APP-CT | loop PS1 | 2303 ± 46 | P = 0.001 |
| 500 μM aspirin (n = 11) | APP-CT | loop PS1 | 2058 ± 45 | ns |

*One-way ANOVA;
ns: not significant.

NSAIDs Lead to a Change in PS1 Conformation

We next adapted the FLIM assay to directly test the possibility that NSAIDs alter presenilin conformation. We examined a series of epitopes on PS1 to find a pair that consistently showed a close association in order to develop an assay of PS1 conformation. We immunostained the N-terminus (PS1-NT) and the C-terminus of PS1 (PS1-CT) with a FITC and Cy3-labeled antibody respectively. In the presence of an acceptor attached to the PS1-CT, FITC lifetime was shortened to ˜1800 psec, suggesting that the N- and C-termini come in close proximity (Annaert, W. G. et al., Neuron 32, 579-589, 2001). As a negative control, we double immunostained cells for PS1 and an ER-resident protein known to colocalize with PS1, Bip (glucose-regulated protein 78). Despite strong colocalization of PS1 and Bip in the ER, there was no change in FITC lifetime, indicating that these two proteins are not in close enough proximity to support FRET. We used this assay to monitor PS1 conformation and to test how different agents impact its conformation. We treated PS70 cells with different NSAIDs or a vehicle (DMSO or ethanol, 0.1%) for 24 hours and stained the PS1-NT and the PS1-CT with FITC- and Cy3-labeled antibodies respectively. As expected, FITC lifetime was shortened in cells stained for both N- and C-termini of PS1 (Table 6). FITC lifetime was significantly longer after treatment of cells with 375 μM S-ibuprofen, 150 μM indomethacin or 375 μM flurbiprofen reflecting an increase in the distance between the N- and C-termini of PS1. No statistically significant change was observed after treatment with 500 μM aspirin or 400 μM naproxen.

Thus, NSAIDs that reduce Aβ₄₂ alter the conformation of PS1, whereas those that do not alter Aβ₄₂, do not change PS1 conformation.

Table 6 illustrates that NSAIDS that markedly decrease Aβ₄₂ also change PS1 conformation assessed by FLIM. Table 6 summarizes the data from the FLIM assay of proximity between the N- and C-termini of PS1. Cells were immunostained with a FITC and Cy3-labeled antibody against the N- and C-termini respectively. In the absence of a FRET acceptor FITC lifetime is ˜2400 psec. In the presence of a FRET acceptor, Cy3 against the C-terminus, FITC lifetime is shortened to ˜1900 psec. After treatment with ibuprofen, indomethacin or flurbiprofen, but not naproxen, aspirin, DAPT or WPE-III-31C, FITC lifetime is significantly increased reflecting an increase in the distance between the N- and C-termini of PS1. A representative experiment is shown (total number of experiments=12).

TABLE 6

| | FRET | FRET | FITC lifetime | P* value |
|---|---|---|---|---|
| | donor | acceptor | PS1-NT | compared |
| Condition | FITC-labeled | Cy3-labeled | (mean ± SD) | to vehicle |
| DMSO (n = 47) | PS1-NT | PS1-CT | 1868 ± 266 | — |
| 375 μM S-ibuprofen (n = 18) | PS1-NT | PS1-CT | 2191 ± 104 | P < 0.001 |
| 400 μM naproxen (n = 13) | PS1-NT | PS1-CT | 1895 ± 385 | ns |
| 150 μM indomethacin (n = 9) | PS1-NT | PS1-CT | 2170 ± 110 | P < 0.001 |
| 500 μM aspirin (n = 6) | PS1-NT | PS1-CT | 2038 ± 284 | ns |
| 375 μM flurbiprofen (n = 12) | PS1-NT | PS1-CT | 2094 ± 185 | P = 0.04 |
| 1 μM DAPT (n = 15) | PS1-NT | PS1-CT | 1959 ± 247 | ns |
| 1 μM WPE-III-31C (n = 14) | PS1-NT | PS1-CT | 1985 ± 205 | ns |

*One-way ANOVA;
ns: not significant.

To verify that the observed changes were not cell-type specific we tested our findings in primary cultured neurons. After treatment with ibuprofen or indomethacin but not naproxen, FITC lifetime was significantly increased reflecting an increase in the distance between the N- and C-termini of PS1.

We also tested whether the same effect was observed using γ-secretase inhibitors, such as DAPT or WPE-III-31C (Kornilova, A. Y., et al., J. Biol. Chem. 278, 16470-16473, 2003; Esler, W. P. et al., Proc. Natl. Acad. Sci. USA 99, 2720-2725, 2002), which are known to inhibit Aβ production. We treated PS70 cells with 1 μM DAPT or 1 μM WPE-III-31C prior to FLIM analysis; no change in FITC lifetime was observed with either γ-secretase inhibitor, supporting the notion that NSAIDs behave differently than conventional γ-secretase inhibitors. One of the advantages of the FLIM technique is that it allows spatial analysis of FRETing populations. The FRETing molecules can be displayed in a pseudo-colored image. If the two fluorophores are in close proximity (<10 nm) the color is closer to red, but if they are far apart (no FRET) the color is closer to blue. In cells treated with DMSO (0.1%) or inactive NSAIDs the N- and C-termini of PS1 remain in close proximity reflected as red pixels. However, after treatment with 375 μM ibuprofen, the distance between epitopes increases as indicated by the shift towards the blue color.

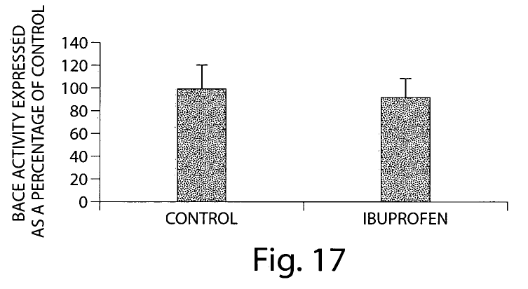

To address whether the effect of NSAIDs on PS1 conformation was observed in vivo at clinically relevant doses of ibuprofen we treated a group of 3-month-old wild type PS1 transgenic mice with S-ibuprofen 375 ppm or a control diet for 15 days (Lim, G. P. et al., J. Neurosci. 20, 5709-5714, 2000; Jantzen, P. T. et al., J. Neurosci. 22, 2246-2254, 2002). The brain levels of Aβ₄₂ and Aβ₄₀ were measured by ELISA and we found that, as previously reported (Weggen, S. et al., Nature 414, 212-216, 2001; Lim, G. P. et al., J. Neurosci. 20, 5709-5714, 2000; Jantzen, P. T. et al., J. Neurosci. 22, 2246-2254, 2002; and Eriksen, J. L. et al., J. Clin. Invest. 112, 440-449, 2003), administration of ibuprofen resulted in a decrease in the Aβ₄₂/Aβ40 ratio in the brain compared with that of control diet-treated animals (12% reduction, n=12, P=0.03). We also measured BACE activity using a BACE activity assay (Fukumoto, H., et al., Am. J. Pathol. 164, 719-725, 2004), and found no differences between the two groups (see FIG. 17). Half of the brain was fixed, sectioned at 35 μm, and immunostained with antibodies against the N- and C-termini of PS1. As expected FITC lifetime was shortened in sections stained for both N- and C-termini (1934±161 psec) compared with FITC without a FRET acceptor. FITC lifetime was significantly longer in sections of mice treated with ibuprofen compared with that of control diet (2205±104 psec, n=12, P=0.02). These results strongly indicate the physiological relevance of our cell-based studies.

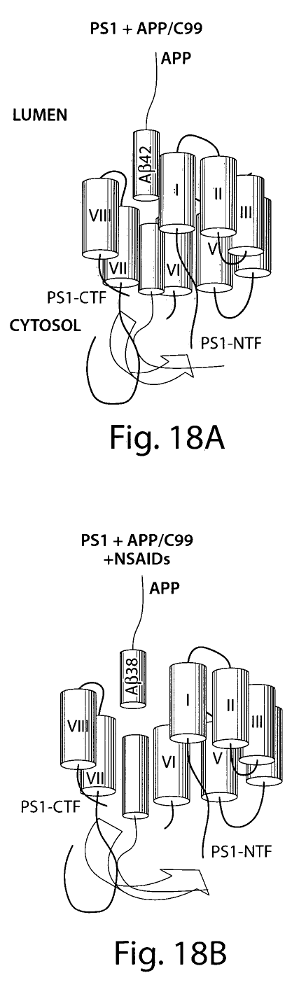

Our data were consistent with a model (FIG. 18) in which NSAIDs allosterically alter PS1 conformation, leading to a change in how APP is presented to γ-secretase for cleavage. FIG. 18 shows a schematic of a model for the effect of NSAIDs on presenilin conformation. FIG. 18A shows PS1 as a ring-like structure that cleaves APP/C99 through the two conserved aspartates (black dots) located in TM6 and 7, liberating Aβ42. FIG. 18B shows that in the presence of NSAIDs, the distance between the N- and C-termini of PS1 increases. This change in the conformation of PS1 shifts the cleavage of APP/C99 towards shorter Aβ species like Aβ38.

Equivalents

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1. A method of assessing the distance between two molecules comprising:
obtaining a biological sample comprising a first molecule labeled with a donor fluorophore and a second molecule labeled with a acceptor fluorophore,
exposing the biological sample to a pulsed excitation source with a predetermined pulse rate and pulse frequency,
measuring the fluorescence lifetime of the donor fluorophore,
calculating the distance between the donor and acceptor fluorophores as a measure of the distance between the first and second molecules, wherein the length of the fluorescence lifetime correlates with the distance between the first and second molecules,
spatially resolving the proportion of donor fluorophore that transfers fluorescence energy, and optionally
imaging the biological sample.

2-25. (Cancelled)

26. A method of determining a molecule's conformation comprising:
obtaining a biological sample comprising a molecule labeled in a first region with a donor fluorophore and labeled in second region with an acceptor fluorophore,
exposing the biological sample to a pulsed excitation source with a predetermined pulse rate and pulse frequency,
measuring the fluorescence lifetime of the donor fluorophore, and
calculating the distance between the donor and acceptor fluorophores as a determination of the conformation of the molecule, wherein the length of the fluorescence lifetime correlates with the distance between the first and second molecule regions, spatially resolving the proportion of donor fluorophore that transfers fluorescence energy, and optionally
imaging the biological sample.

27-51. (Cancelled)

52. A method of assessing the distance between presenilin 1 and APP comprising:
obtaining a biological sample comprising one of presenilin 1 or APP labeled with a donor fluorophore and the other of presenilin 1 or APP labeled with a acceptor fluorophore,
exposing the biological sample to a pulsed excitation source with a predetermined pulse rate and pulse frequency,
measuring the fluorescence lifetime of the donor fluorophore,
calculating the distance between the donor and acceptor fluorophores as a measure of the distance between the presenilin 1 and APP molecules, wherein the length of the fluorescence lifetime correlates with the distance between the presenilin 1 and APP molecules,
spatially resolving the proportion of donor fluorophore that transfers fluorescence energy, and optionally
imaging the biological sample.

53. The method of claim 52, wherein the imaging is light microscopy imaging.

54. The method of claim 52, wherein the biological sample comprises cells.

55. The method of claim 54, wherein the cells are transformed cells.

56. The method of claim 54, wherein the cells are live cells.

57. The method of claim 54, wherein the cells are fixed cells.

58. The method of claim 54, wherein the cells are neuronal cells.

59. The method of claim 52, wherein the donor fluorophore has an emission spectrum that overlaps with the excitation spectrum of the acceptor fluorophore.

60. The method of claim 52, wherein the donor fluorophore is selected from the group that includes: fluorescein isothiocyanate (FITC), Alexa 488, Oregon Green 514, and Oregon Green 488.

61. The method of claim 60, wherein the donor fluorophore is fluorescein isothiocyanate (FITC).

62. The method of claim 52, wherein the acceptor fluorophore is selected from the group consisting of: Cy3, Rhodamine, Texas Red, and Alexa 568.

63. The method of claim 62, wherein the acceptor fluorophore is Cy3.

64. The method of claim 52, wherein the presenilin 1 and APP molecules are labeled using a means selected from the group consisting of: antibody labeling, derivatization, chemical modification, and genetic engineering.

65. The method of claim 52, wherein the pulsed excitation source is a laser.

66. The method of claim 65, wherein the laser is a Ti-sapphire laser.

67. The method of claim 52, wherein the predetermined pulse length is from about 1 femtosecond to about 1 picosecond.

68. The method of claim 67, wherein the predetermined pulse length is about 1 femtosecond.

69. The method of claim 67, wherein the predetermined pulse length is about 10 femtoseconds.

70. The method of claim 67, wherein the predetermined pulse length is about 100 femtoseconds.

71. The method of claim 52, wherein the predetermined pulse frequency is from about one pulse per 1 nanosecond to about 1 pulse per 100 nanoseconds.

72. The method of claim 71, wherein the predetermined pulse frequency is about one pulse per 12 nanoseconds.

73. A method of screening a candidate pharmaceutical agent for an effect on the distance between a presenilin 1 molecule and an APP molecule comprising:
contacting a first biological sample with a candidate pharmaceutical agent and assessing the distance between a presenilin 1 molecule and an APP molecule in the sample as in the method of claim 52,
assessing the distance between a presenilin 1 molecule and an APP molecule in a second biological sample as in the method of claim 52, wherein the second biological sample is a sample not contacted with the candidate pharmaceutical agent,
comparing the distance between the presenilin 1 and APP molecules in the first biological sample and between the presenilin 1 and APP molecules in the second biological sample as an indication of the effect of the candidate pharmaceutical agent on the distance between the presenilin 1 and APP molecules.

74. A method of determining a molecule's conformation comprising:
obtaining a biological sample comprising a presenilin 1 molecule labeled in a first region with a donor fluorophore and labeled in second region with an acceptor fluorophore,
exposing the biological sample to a pulsed excitation source with a predetermined pulse rate and pulse frequency,
measuring the fluorescence lifetime of the donor fluorophore, and
calculating the distance between the donor and acceptor fluorophores as a determination of the conformation of the presenilin 1 molecule, wherein the length of the fluorescence lifetime correlates with the distance between the first and second molecule regions,
spatially resolving the proportion of donor fluorophore that transfers fluorescence energy, and optionally
imaging the biological sample.

75. The method of claim 74, wherein the imaging is light microscopy imaging.

76. The method of claim 74, wherein the biological sample comprises cells.

77. The method of claim 76, wherein the cells are transformed cells.

78. The method of claim 76, wherein the cells are live cells.

79. The method of claim 76, wherein the cells are fixed cells.

80. The method of claim 76, wherein the cells are neuronal cells.

81. The method of claim 74, wherein the first and second regions are selected from N-terminal region, mid-region or C-terminal region of the protein.

82. The method of claim 74, wherein the donor fluorophore has an emission spectrum that overlaps with the excitation spectrum of the acceptor fluorophore.

83. The method of claim 74, wherein the donor fluorophore is selected from the group that includes: fluorescein isothiocyanate (FITC), Alexa 488, Oregon Green 514, and Oregon Green 488.

84. The method of claim 83, wherein the donor fluorophore is fluorescein isothiocyanate (FITC).

85. The method of claim 74, wherein the acceptor fluorophore is selected from the group consisting of: Cy3, Rhodamine, Texas Red, and Alexa 568.

86. The method of claim 85, wherein the acceptor fluorophore is Cy3.

87. The method of claim 74, wherein the molecule regions are labeled using a means selected from the group consisting of: antibody labeling, derivatization, chemical modification, and genetic engineering.

88. The method of claim 74, wherein the pulsed excitation source is a laser.

89. The method of claim 88, wherein the laser is a Ti-sapphire laser.

90. The method of claim 74, wherein the predetermined pulse length is from about 1 femtosecond to about 1 picosecond.

91. The method of claim 90, wherein the predetermined pulse length is about 1 femtosecond.

92. The method of claim 90, wherein the predetermined pulse length is about 10 femtoseconds.

93. The method of claim 90, wherein the predetermined pulse length is about 100 femtoseconds.

94. The method of claim 74, wherein the predetermined pulse frequency is from about one pulse per 1 nanosecond to about 1 pulse per 100 nanoseconds.

95. The method of claim 94, wherein the predetermined pulse frequency is about one pulse per 12 nanoseconds.

96. A method of screening a candidate pharmaceutical agent for an effect on the conformation of a presenilin 1 molecule comprising:
contacting a first biological sample with a candidate pharmaceutical agent and assessing the conformation of a presenilin 1 molecule using the method of claim 74,
assessing the conformation of the presenilin 1 molecule in a second biological sample using the method of claim 74, wherein the second biological sample is a sample not contacted with the candidate pharmaceutical agent,
comparing the conformation of the presenilin 1 molecule in the first biological sample and conformation of the presenilin 1 molecule in the second biological sample as an indication of the effect of the candidate pharmaceutical agent on the conformation of the presenilin 1 molecule.
